(19) United States
(12) Patent Application Publication
(10) Pub. No.: US 2003/0157592 A1
(43) Pub. Date: Aug. 21, 2003

(54) Moss genes from physcomitrella patens encoding proteins involved in the synthesis of tocopherols and carotenoids

(76) Inventors: Jens Lerchl, Ladenburg (DE); Andreas Renz, Limburgerhof (DE); Thomas Ehrhardt, Speyer (DE); Andreas Reindl, Birkenheide (DE); Petra Cirpus, Mannheim (DE); Friedrich Bischoff, Mannheim (DE); Markus Frank, Ludwigshafen (DE); Annette Freund, Limburgerhof (DE); Elke Duwenig, Ludwigshafen (DE); Ralf-Michael Schmidt, Kirrweiler (DE); Ralf Reski, Oberried (DE); Ralf Badur, Goslar (DE)

Correspondence Address: KEIL & WEINKAUF, 1350 CONNECTICUT AVENUE, N.W., WASHINGTON, DC, 20036, US

(21) Appl. No.: 10/149,759

(22) PCT Filed: Dec. 14, 2000
(86) PCT No.: PCT/EP00/12698

(30) Foreign Application Priority Data
Dec. 16, 1999 (US) 60171121

(51) Int. Cl. C12P 23/00; C12N 9/10; A01H 1/00; C12N 15/82; C12P 21/02; C12N 5/04

(52) U.S. Cl. 435/67; 435/69.1; 435/193; 435/320.1; 435/419; 800/278; 800/282

(57) ABSTRACT

Isolated nucleic acid molecules, designated TCMRP nucleic acid molecules, which encode novel TCMRPs from e.g. Phycomitrella patens are described. The invention also provides antisense nucleic acid molecules, recombinant expression vectors containing TCMRP nucleic acid molecules, and host cells into which the expression vectors have been introduced. The invention still further provides isolated TCMRPs, mutated TCMRPs, fusion proteins, antigenic peptides and methods for the improvement of production of a desired compound from transformed cells, organisms or plants based on genetic engineering of TCMRP genes in these organisms.

### BACKGROUND OF THE INVENTION

Certain products and by-products of naturally-occurring metabolic processes in cells have utility in a wide array of industries, including the food, feed, cosmetics, and pharmaceutical industries. These molecules, collectively termed ‘fine chemicals’, include organic acids, both proteinogenic and non-proteinogenic amino acids, nucleotides and nucleosides, lipids and fatty acids, carotenoids, diols, carbohydrates, aromatic compounds, vitamins and cofactors and enzymes.

Their production is most conveniently performed through the large-scale culture of bacteria developed to produce and secrete large quantities of one or more desired molecules. One particularly useful organism for this purpose is Corynebacterium glutamicum, a gram positive, nonpathogenic bacterium.

Through strain selection, a number of mutant strains of the respective microorganisms have been developed which produce an array of desirable compounds. However, selection of strains improved for the production of a particular molecule is a time-consuming and difficult process.

Alternatively the production of fine chemicals can be most conveniently performed via the large scale production of plants developed to produce one of aforementioned fine chemicals. Of particular interest for this purpose are all crop plants for food and feed uses. Increased or modulated compositions of fine chemicals like amino acids, vitamins and nucleotides, in these plants would lead to optimized nutritional qualities.

Through conventional breeding, a number of mutant plants have been developed which produce increased amounts of for example, carotenoids, and amino acids. However, selection of new plant cultivars improved for the production of a particular molecule is a time-consuming and difficult process.

### SUMMARY OF THE INVENTION

This invention provides novel nucleic acid molecules which may be used to modify tocopherols and carotenoids in plants, algae and microorganisms.

The naturally occurring eight compounds with vitamin E activity are derivatives of 6-chromanol (Ullmann's Encyclopedia of Industrial Chemistry, Vol. A 27 (1996), VCH Verlagsgesellschaft, Chapter 4., 478-488, Vitamin E). The group of the tocopherols (1α-δ) has a saturated side chain, while the group of the tocotrienols (2α-δ) has an unsaturated side chain:

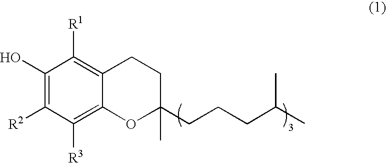

1a, α-tocopherol: R¹=R²=R³CH₃

1b, β-tocopherol: R¹=R³=CH₃, R²=H

1c, γ-tocopherol: R¹=H, R²=R³=CH₃

1d, δ-tocopherol: R¹=R²=H R³ =CH₃

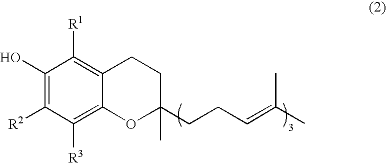

2b, α-tocotrienol: R¹=R²=R³ CH₃

2b, β-tocotrienol: R¹=R³=CH₃, R=H

2b, γ-tocotrienol: R¹=H, R²=R³ =CH₃

2b, δ-tocotrienol: R¹=R²=H, R³CH₃

In the present invention, tocopherols are to be understood as meaning all the abovementioned tocopherols and tocotrienols and derivates thereof with vitamin E activity.

These compounds with vitamin E activity (vitamin E compounds) are important natural lipid-soluble substances, which among other activities have especially the function of antioxidants. A lack of vitamin E in humans and animals leads to pathophysiological situations. Vitamin E compounds therefore have an important economical value as additives in the food and feed sectors, in pharmaceutical formulations and in cosmetic applications.

An economical method for the production of vitamin E compounds, and foodstuffs and animal feeds with an elevated vitamin E content are therefore of great importance.

WO 00/10380 describes the gene sequence encoding the 2-methyl-6-phytylplastoquinol-methyltransferase from the prokaryotic organism Synechocystis spec. PCC6803. WO 97/27285 describes the mapping of the gene locus of p-hydroxyphenylpyruvate dioxygenase encoding gene of Arabidopsis thaliana. Speculations are done about the effects of overexpression or downregulation of the plant enzyme on the vitamin E content or herbicide resistance in transgenic plants. WO 99/04622 and D. DellaPenna et al., Science 1998, 282, 2098-2100 describe gene sequences encoding a γ-tocopherol methyltransferase from Synechocystis PCC6803 and Arabidopsis thaliana and their incorporation into plants. However, the transgenic plants show only a shift in the spectum of tocopherols, i.e. a shift from gamma-tocopherol to alpha-tocopherol because of the higher expression of γ-tocopherol methyltransferase. No data are shown concerning a higher yield of tocopherols, i. e. a quantitative improvement in tocopherol content.

To date no economical methods are available for an effective production of tocopherols and/or carotinoids in transgenic organisms, i. e. for effectively increasing the metabolite flow in the direction of increased tocopherol and/or carotinoid content in transgenic organisms, for example in transgenic plants, by overexpressing one or several biosynthesis genes, alone or in any combination, related to the tocopherol and/or carotinoid metabolism.

Methods which are particularly economical are biotechnological methods which exploit proteins and biosynthesis genes from tocopherol or carotinoid biosynthesis from organisms producing these compounds.

Microorganisms like Corynebacterium and fungi and algae like Phaeodactylum are commonly used in industry for the large-scale production of a variety of fine chemicals.

Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals. This improved production or efficiency of production of a fine chemical may be due to a direct effect of manipulation of a gene of the invention, or it may be due to an indirect effect of such manipulation.

Given the availability of cloning vectors and techniques for genetic manipulation of ciliates such as disclosed in WO9801572 or algae and related organisms such as Phaeodactylum tricornutum (described in Falciatore et al., 1999, Marine Biotechnology 1 (3):239-251 as well as Dunahay et al. 1995, Genetic transformation of diatoms, J. Phycol. 31:10004-1012 and references therein) the nucleic acid molecules of the invention may be utilized in the genetic engineering of these organisms to make them better or more efficient producers of one or more fine chemicals. This improved production or efficiency of production of a fine chemical may be due to a direct effect of manipulation of a gene of the invention, or it may be due to an indirect effect of such manipulation.

The moss Physcomitrella patens represents one member of the mosses. It is related to other mosses such as Ceratodon purpureus which is capable to grow in the absense of light. Further Physcomitrella patens represents the only plant organism which can be utilized for targeted disruption of genes by homologous recombination. Mutants generated by this technique are useful to characterize the function for genes described in the invention. Mosses like Ceratodon and Physcomitrella share a high degree of homology on the DNA sequence and polypeptide level allowing the use of heterologous screening of DNA molecules with probes evolving from other mosses or organisms, thus enabling the derivation of a consensus sequence suitable for heterologous screening or functional annotation and prediction of gene functions in third species. The ability to identify such functions can therefor have significant relevance, e.g., prediction of substrate specificity of enzymes. Further, these nucleic acid molecules may serve as reference points for the mapping of moss genomes, or of genomes of related organisms.

This invention provides novel nucleic acid molecules which encode proteins, referred to herein as Tocopherol, and Carotenoid Metabolism Related Proteins (TCMRP). These TCMRPs are capable of, for example, performing an enzymatic step involved in the metabolism of certain fine chemicals, including tocopherols and/or carotenoids.

Given the availability of cloning vectors for use in plants and plant transformation, such as those published in and cited therein: Plant Molecular Biology and Biotechnology (CRC Press, Boca Raton, Fla.), chapter 6/7, S.71-119 (1993); F. F. White, Vectors for Gene Transfer in Higher Plants; in: Transgenic Plants, Vol. 1, Engineering and Utilization, eds.: Kung und R. Wu, Academic Press, 1993, 15-38; B. Jenes et al., Techniques for Gene Transfer, in: Transgenic Plants, Vol. 1, Engineering and Utilization, eds.: Kung und R. Wu, Academic Press (1993), 128-143; Potrykus, Annu. Rev. Plant Physiol. Plant Molec. Biol. 42 (1991), 205-225)) the nucleic acid molecules of the invention may be utilized in the genetic engineering of a wide variety of plants to make it a better or more efficient producer of one or more fine chemicals. This improved production or efficiency of production of a fine chemical may be due to a direct effect of manipulation of a gene of the invention, or it may be due to an indirect effect of such manipulation.

There are a number of mechanisms by which the alteration of an TCMRP of the invention may directly affect the yield, production, and/or efficiency of production of a fine chemical in plant due to such an altered protein.

The nucleic acid and protein molecules of the invention may directly improve the production or efficiency of production of one or more desired fine chemicals from microorganisms and plants. Using recombinant genetic techniques well known in the art, one or more of the biosynthetic or degradative enzymes of the invention for tocopherols and/or carotinoids may be manipulated such that its function is modulated. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell.

Further, one gene or one enzyme of the invention for tocopherols and/or carotinoids or preferably a combination of several genes or enzymes of the invention can be transformed into host cells (e. g. starting organism or already genetically modified host system), whereby the gene(s) or enzyme(s) can be modified either in their activity or number in the correponding host cell (e.g. plant). Besides, the host cell itself might be already genetically manipulated (e.g. in key position of the pathway) in the way that the flux of metabolites can be directed to higher yields of tocopherols and/or carotinoids, when the cell is used to be transformed with one or more genes (encoding the corresonding enzymes) of the invention for tocopherols and/or carotinoids. In each case, the overall yield or rate of production of the desired fine chemical may be increased. In one preferred embodiment of the instant invention the genes encoding the TCMR proteins γ-tocopherol-methyltransferase (gamma-TMT type I), 2-methyl-6-phytylplastoquinol methyltransferase (gamma-TMT type II) and/or 4-hydroxyphenylpyruvate dioxygenase alone or in any combination have a substancial effect on the production of the desired fine chemical, preferred vitamin E compounds or in the production of relevant precursors, e.g. tocopherol precursors such as homogentisic acid and/or phytylpyrophosphate and/or geranylgeranyl-pyrophosphate. In the instant invention, the genes encoding these enzymes mentioned above, i.e. γ-tocopherol-methyltransferase (gamma-TMT type I), 2-methyl-6-phytylplastoquinol methyltransferase (gamma-TMT type II) and/or 4-hydroxyphenylpyruvate dioxygenase, can be isolated from the moss Physcomitrella patens and transferred into suitable host cells, but the invention is not limited to this organism as a source for the nucleic acid isolation. Thus, the mentioned genes and/or enzymes can also be isolated from any other organisms, e.g. prokaryotes or eukaryotes, which comprises an endogenous sequence mentioned above. Preferred examples for such organisms, especially in view to the enzyme 4-hydroxyphenylpyruvate dioxygenase, are Streptomyces avermitilis (database accession number of the corresponding gene is AL 096852), Rattus norwegicus (database accession number AF 082834), Synechocystis spec. PCC6803 or Arabidopsis thaliana (DellaPenna, D. et al., 1998, Science, 282, 2098-2100).

It is also possible that alterations in the protein and nucleotide molecules of the invention may improve the production of other fine chemicals besides the tocopherols and/or carotinoids through indirect mechanisms. Metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the proteins of the invention, the production or efficiency of activity of another fine chemical biosynthetic or degradative pathway may be impacted. For example, amino acids serve as the structural units of all proteins, yet may be present intracellularly in levels which are limiting for protein synthesis; therefore, by increasing the efficiency of production or the yields of one or more amino acids within the cell, proteins, such as biosynthetic or degradative proteins, may be more readily synthesized. Likewise, an alteration in a metabolic pathway enzyme such that a particular side reaction becomes more or less favored may result in the over- or under-production of one or more compounds which are utilized as intermediates or substrates for the production of a desired fine chemical.

Those TCMRPs involved in the transport of fine chemical molecules from the cell may be increased in number or activity such that greater quantities of these compounds are allocated to different plant cell compartments or the cell exterior space from which they are more readily recovered and partitioned into the biosynthetic flux or deposited. Similarly, those TCMRPs involved in the import of nutrients necessary for the biosynthesis of one or more fine chemicals (e.g. tocopherols and/or carotinoids) may be increased in number or activity such that these precursors, cofactors, or intennediate compounds are increased in concentration within the cell or within the storing compartments. The invention pertains to an isolated nucleic acid molecule which encodes an TCMRP or an TCMRP polypeptide involved in assisting in transmembrane transport.

The mutagenesis of one or more TCMRPs of the invention may also result in TCMRPs having altered activities which indirectly impact the production of one or more desired fine chemicals from plants. For example, TCMRPs of the invention involved in the export of waste products may be increased in number or activity such that the normal metabolic wastes of the cell (possibly increased in quantity due to the overproduction of the desired fine chemical) are efficiently exported before they are able to damage nucleic acids and proteins within the cell (which would decrease the viability of the cell) or to interfere with fine chemical biosynthetic pathways (which would decrease the yield, production, or efficiency of production of the desired fine chemical). Further, the relatively large intracellular quantities of the desired fine chemical may in itself be toxic to the cell or may interfere with enzyme feedback mechanisms such as allosteric regulation, so by increasing the activity or number of transporters able to export this compound from the compartment, one may increase the viability of seed cells, in turn leading to a greater number of cells in the culture producing the desired fine chemical. The TCMRPs of the invention may also be manipulated such that the relative amounts of different tocopherols and/or carotinoids are produced. This can be appreciable for optimizing plant nutritional composition. In plants these changes can moreover also influence other characteristic like tolerance towards abiotic and biotic stress conditions.

This invention provides novel nucleic acid molecules which encode TCMRPs, which are capable of, for example, performing an enzymatic step involved in the metabolism of molecules important for the normal functioning of cells, such as tocopherols and/or carotinoids. Nucleic acid molecules encoding an TCMRP are referred to herein as TCMRP nucleic acid molecules. In a preferred embodiment, the TCMRP performs an enzymatic step related to the metabolism of one or more tocopherols and/or carotinoids. Examples of such proteins include those encoded by the genes set forth in the Appendix A and B and Table 1.

As biotic and abiotic stress tolerance is a general trait wished to be inherited into a wide variety of plants like maize, wheat, rye, oat, triticale, rice, barley, sorghum, potato, tomato, soyabean, bean, pea, peanut, cotton, rapeseed, canola, alfalfa, grape, fruit plants (apple, pear, pinapple), bushy plants (coffee, cacao, tea), trees (oil palm, coconut), legumes, perennial grasses, and forage crops. These crops plants are also preferred target plants for a genetic engineering as one further embodiment of the present invention. More preferably are corp plants and oil seed plants and most preferably are rape and soyabean.

The nucleic acid constructs according to the invention can be used for the generation of genetically modified organisms, hereinbelow also termed transgenic organisms.

Starting or host organisms are to be understood as meaning prokaryotic or eukaryotic organisms such as, for example, microorganisms, mosses or plants. Preferred micororganisms are bacteria, yeasts, algae or fungi. In one preferred embodiment of the instant invention host organisms are plants.

Examples of preferred plants are Tagetes, sunflowers, Arabidopsis, tobacco, red pepper, soyabeans, tomatoes, aubergines, capsicums, carrots, potatoes, maize, saladings and cabbages, cereals, alfalfa, oats, barley, rye, wheat, Triticale, panic grasses, rice, luceme, flax, cotton, hemp, Brassicaceae such as, for example, oilseed rape or canola, sugar beet, sugar cane, nut and grapevine species or woody species such as, for example, aspen or yew. More preferably are crop plants or oil seed plants, most preferably are Arabidopsis thaliana, Tagetes erecta, Brassica napus, Nicotiana tabacum, canola or potatoes. Especially preferred are rape or soyabeans.

Genetically modified or transgenic organisms are to be understood as meaning the corresponding transformed starting organisms.

The invention relates to a genetically modified organism where the genetic modification of the gene expression of a nucleic acid according to the invention relative to a wild type is increased in the event that the starting organism comprises a nucleic acid according to the invention or caused in the event that the starting organism does not contain a nucleic acid according to the invention.

Transgenic organisms comprising at least one exogenous or at least one additional endogenous gene according to the invention which already in the form of the starting organisms possess the biosynthesis genes for the production of tocopherols such as, for example, plants or other photosynthetically active organisms such as, for example, cyanobacteria, mosses or algae exhibit an increased tocopherol content compared with the respective wild type or starting organism.

Accordingly, the invention furthermore relates to genetically modified organisms, wherein the genetically modified organism exhibits an increased tocopherol content relative to the wild type in the case where the starting organism is capable of producing tocopherols, or is capable of producing tocopherols in the case where the starting organism comprises the genes required for tocopherol biosynthesis.

The invention preferably relates to an above-described genetically modified organism which exhibits an increased tocopherols content over the wild type.

Used in a preferred embodiment as organisms and for the generation of organisms with an increased tocopherols content compared with the wild type are plants, not only as starting organisms but also, accordingly, as genetically modified organisms.

The present invention therefore also relates to processes for the production of tocopherols by growing a genetically modified organism according to the invention, preferably a genetically modified plant according to the invention, which exhibits an increased tocopherol content over the wild type, harvesting the organism and subsequently isolating the tocopherol compounds from the organism.

Genetically modified plants according to the invention with an increased tocopherol content which can be consumed by humans and animals can also be used as foodstuffs or feeds for example directly or after processing which is known per se.

The invention furthermore relates to a method for the generation of genetically modified organisms by introducing a nucleic acid according to the invention or a nucleic acid construct according to the invention into the genome of the starting organism.

Accordingly, one aspect of the invention pertains to isolated nucleic acid molecules (e.g., cDNAs) comprising a nucleotide sequence encoding an TCMRP or biologically active portions thereof, as well as nucleic acid fragments suitable as primers or hybridization probes for the detection or amplification of TCMRP-encoding nucleic acid (e.g., DNA or mRNA). In another embodiment, the isolated nucleic acid molecule is at least 15 nucleotides in length and hybridizes under stringent conditions to a nucleic acid molecule comprising a nucleotide sequence of Appendix A. Preferably, the isolated nucleic acid molecule corresponds to a naturally-occurring nucleic acid molecule. More preferably, the isolated nucleic acid encodes a naturally-occurring Physcomitrella patens TCMRP, or a biologically active portion thereof. In particularly preferred embodiments, the isolated nucleic acid molecule comprises one of the nucleotide sequences set forth in Appendix A or the coding region or a complement thereof of one of these nucleotide sequences. In other particularly preferred embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence set forth in Appendix A, or a portion thereof. In other preferred embodiments, the isolated nucleic acid molecule encodes one of the amino acid sequences set forth in Appendix B. The preferred TCMRP of the present invention also preferably possess at least one of the TCMRP activities described herein.

In another embodiment, the instant nucleic acid molecule is full length or nearly full length nucleic acid molecule with an homology of at least about 50%, preferably at least about 60%, more preferably at least about 70%, 80% or 90%, and even more preferably at least about 95%, 96%, 97%, 98%, 99% or more homologous to a nucleotide sequence set forth in Appendix A.

In another embodiment, the isolated nucleic acid molecule encodes a protein or portion thereof wherein the protein or portion thereof includes an amino acid sequence which is sufficiently homologous to an amino acid sequence of Appendix B, e.g., sufficiently homologous to an amino acid sequence of Appendix B such that the protein or portion thereof maintains an TCMRP activity. Preferably, the protein or portion thereof encoded by the nucleic acid molecule maintains the ability to perform an enzymatic reaction in a tocopherol and/or carotinoid metabolic pathway. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (e.g., an entire amino acid sequence selected from those sequences set forth in Appendix B). In another preferred embodiment, the protein is a full length or nearly full length Physcomitrella patens protein is substantially homologous to an entire amino acid sequence of Appendix B (encoded by an open reading frame shown in Appendix A). As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

In another preferred embodiment, the isolated nucleic acid molecule is derived from Physcomitrella patens and encodes a protein (e.g., an TCMRP fusion protein) which includes a biologically active domain which is at least about 50% or more homologous to one of the amino acid sequences of Appendix B and is able to perform an enzymatic reaction in a tocopherol and/or carotinoid metabolic pathway or has one or more of the activities set forth in Table 1, and which also includes heterologous nucleic acid sequences encoding a heterologous polypeptide or regulatory regions.

Preferably, so-called conservative exchanges are carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Preferred positions for deletions are the termini of the polypeptide and the linkages between the individual protein domains.

Insertions are introductions of amino acids into the polypeptide chain, a direct bond formally being replaced by one or more amino acids.

One embodiment of the invention pertains to TCMRP polypeptides, where by of one or more amino acids are substituted or exchanged by one or more amino acids.

Another aspect of the invention pertains to an TCMRP polypeptide whose amino acid sequence can be modulated with the help of art-known computer simulation programms resulting in an polypeptide with e.g. improved activity or altered regulation (molecular modelling). On the basis of this artificially generated polypeptide sequences, a corresponding nucleic acid molecule coding for such a modulated polypeptide can be synthesized in-vitro using the specific codon-usage of the desired host cell, e.g. of microorganisms, mosses, algae, ciliates, fungi or plants (back-translated nucleic acid sequences). In a preferred embodiment, even these artificial nucleic acid molecules coding for improved TCMRP proteins are within the scope of this invention.

Another aspect of the invention pertains to vectors, e.g., recombinant expression vectors, containing the nucleic acid molecules of the invention, and host cells into which such vectors have been introduced, especially microorganims, plant cells, plant tissue, organs or whole plants. In one embodiment, such a host cell is a cell capable of storing fine chemical compounds in order to isolate the desired compound from harvested material. The compound or the TCMRP can then be isolated from the medium or the host cell, which in plants are cells containing and storing fine chemical compounds, most preferably cells of storage tissues like epidermal and seed cells.

Yet another aspect of the invention pertains to a genetically altered Physcomitrella patens plant in which an TCMRP gene has been introduced or altered. In one embodiment, the genome of the Physcomitrella patens plant has been altered by introduction of a nucleic acid molecule of the invention encoding wild-type or mutated TCMRP sequence as a transgene. In another embodiment, an endogenous TCMRP gene within the genome of the Physcomitrella patens plant has been altered, e.g., functionally disrupted, by homologous recombination with an altered TCMRP gene. In a preferred embodiment, the plant organism belongs to the genus Physcomitrella or Ceratodon, with Physcomitrella being particularly preferred. In a preferred embodiment, the Physcomitrella patens plant is also utilized for the production of a desired compound, such as tocopherols and/or carotinoids. Hence in another preferred embodiment, the moss Physcomitrella patens can be used to show the function of new, yet unidentified genes of mosses or plants using homologous recombination based on the nucleic acids described in this invention.

Still another aspect of the invention pertains to an isolated TCMRP or a portion, e.g., a biologically active portion, thereof. In a preferred embodiment, the isolated TCMRP or portion thereof can catalyze an enzymatic reaction involved in one or more pathways for the metabolism of tocopherols and/or carotinoids. In another preferred embodiment, the isolated TCMRP or portion thereof is sufficiently homologous to an amino acid sequence of Appendix B such that the protein or portion thereof maintains the ability to catalyze an enzymatic reaction involved in one or more pathways for the metabolism of tocopherols and/or carotinoids.

The invention also provides an isolated preparation of an TCMRP. In preferred embodiments, the TCMRP comprises an amino acid sequence of Appendix B. In another preferred embodiment, the invention pertains to an isolated full length protein which is substantially homologous to an entire amino acid sequence of Appendix B (encoded by an open reading frame set forth in Appendix A). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B. In other embodiments, the isolated TCMRP comprises an amino acid sequence which is at least about 50% or more homologous to one of the amino acid sequences of Appendix B and is able to perform an enzymatic reaction in a tocopherol and/or carotinoid metabolic pathway in a microorganism or a plant cell or has one or more of the activities set forth in Table 1.

Alternatively, the isolated TCMRP can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B. It is also preferred that the preferred forms of TCMRP also have one or more of the TCMRP activities described herein.

The TCMRP polypeptide, or a biologically active portion thereof, can be operatively linked to a non-TCMRP polypeptide to form a fusion protein. In preferred embodiments, this fusion protein has an activity which differs from that of the TCMRP alone. In other preferred embodiment, this fusion protein performs an enzymatic reaction in a tocopherol and/or carotinoid metabolic pathway. In particularly preferred embodiments, integration of this fusion protein into a host cell modulates production of a desired compound from the cell. Further, the instant invention pertains to an antibody specifically binding to an MP polypeptide mentioned before or to a portion thereof.

Another aspect of the invention pertains to a test kit comprising a nucleic acid molecule encoding an TCMRP, a portion and/or a complement of this nucleid acid molecule used as probe or primer for identifying and/or cloning further nucleic acid molecules involved in the synthesis of amino acids, vitamins, cofactors, nucloetides and/or nucleosides or assisting in transmembrane transport in other cell types or organisms.

In another embodiment the test kit comprises an TCMRP-antibody for identifying and/or purifying further TCMRP molecules or fragments thereof in other cell types or organisms.

Another aspect of the invention pertains to a method for producing a fine chemical. This method involves either the culturing of a suitable microorganism, algae or culturing plant cells tissues, organs or whole plants containing a vector directing the expression of an TCMRP nucleic acid molecule of the invention, such that a fine chemical is produced. In a preferred embodiment, this method further includes the step of obtaining a cell containing such a vector, in which a cell is transformed with a vector directing the expression of an TCMRP nucleic acid. In another preferred embodiment, this method further includes the step of recovering the fine chemical from the culture. In a particularly preferred embodiment, the cell is from the genus Phaeodactylum, mosses, algae or plants.

Another aspect of the invention pertains to a method for producing a fine chemical which involves the culturing of a suitable host cell whose genomic DNA has been altered by the inclusion of an TCMRP nucleic acid molecule of the invention. Further, the invention pertains to a method for producing a fine chemical which involves the culturing of a suitable host cell whose membrane has been altered by the inclusion of an TCMRP of the invention.

Another aspect of the invention pertains to methods for modulating production of a molecule from a kostcell. Such methods include contacting the cell with an agent which modulates TCMRP activity or TCMRP nucleic acid expression such that a cell associated activity is altered relative to this same activity in the absence of the agent. In a preferred embodiment, the cell is modulated for one or more metabolic pathways for tocopherols and/or carotinoids such that the yields or rate of production of a desired fine chemical by this microorganism is improved. The agent which modulates TCMRP activity can be an agent which stimulates TCMRP activity or TCMRP nucleic acid expression. Examples of agents which stimulate TCMRP activity or TCMRP nucleic acid expression include small molecules, active TCMRPs, and nucleic acids encoding TCMRPs that have been introduced into the cell. Examples of agents which inhibit TCMRP activity or expression include small molecules and antisense TCMRP nucleic acid molecules.

Another aspect of the invention pertains to methods for modulating yields of a desired compound from a cell, involving the introduction of a wild-type or mutant TCMRP gene into a cell, either maintained on a separate plasmid or integrated into the genome of the host cell. If integrated into the genome, such integration can be random, or it can take place by recombination such that the native gene is replaced by the introduced copy, causing the production of the desired compound from the cell to be modulated or by using a gene in trans such as the gene is functionally linked to a functional expression unit containing at least a sequence facilitating the expression of a gene and a sequence facilitating the polyadenylation of a functionally transcribed gene.

In a preferred embodiment, said yields are modified. In another preferred embodiment, said desired chemical is increased while unwanted disturbing compounds can be decreased. In a particularly preferred embodiment, said desired fine chemical is a tocopherols and/or carotinoids.

Another aspect of the invention pertains to the fine chemicals produced by a method described before and the use of the fine chemical or a polypeptide of the invention for the production of another fine chemical.

### DETAILED DESCRIPTION OF THE INVENTION

The present invention provides TCMRP nucleic acid and protein molecules which are involved in the metabolism of tocopherols and/or carotinoids in the moss Physcomitrella patens. The molecules of the invention may be utilized in the production or modulation of fine chemicals in microorganisms, algae and plants either directly (e.g., where overexpression or optimization of a vitamin biosynthesis protein has a direct impact on the yield, production, and/or efficiency of production of the vitamin from modified organims), or may have an indirect impact which nonetheless results in an increase of yield, production, and/or efficiency of production of the desired compound or decrease of undesired compounds (e.g., where modulation of the metabolism of tocopherols and/or carotinoids results in alterations in the yield, production, and/or efficiency of production or the composition of desired compounds within the cells, which in turn may impact the production of one or more other fine chemicals).

Preferred mircroorganisms for the production or modulation of fine chemicals are for example Corynehacterium, Synechocystis spec., Synechococcus spec., Ashbya gossypii, Neurospora crassa, Aspergillus spec., Saccharomyces cerevisiae. Preferred algae for the production or modulation of fine chemicals are Chlorella spec., Crypthecodineum spec., Phylodactenum spec. Preferred plants for the production or modulation of fine chemicals are for example mayor crop plants for example maize, wheat, rye, oat, triticale, rice, barley, sorghum, potato, tomato, soybean, bean, pea, peanut, cotton, rapeseed, canola, alfalfa, grape, fruit plants (apple, pear, pinapple), bushy plants (coffee, cacao, tea), trees (oil palm, coconut), legumes, perennial grasses, and forage crops.

Particularly suited for the production or modulation of lipophilic fine chemicals such as tocopherols and/or carotinoids are oil seed plants containing high amounts of lipid compounds like rapeseed, canola, linseed, soybean and sunflower.

Aspects of the invention are further explicated below.

### Fine Chemicals

The term ‘fine chemical’ is art-recognized and includes molecules produced by an organism which have applications in various industries, such as, but not limited to, the pharmaceutical, agriculture, and cosmetics industries. Such compounds include lipids, fatty acids, vitamins, cofactors and enzymes, both proteinogenic and non-proteinogenic amino acids, purine and pyrimidine bases, nucleosides, and nucleotides (as described e.g. in Kuninaka, A. (1996) Nucleotides and related compounds, p. 561-612, in Biotechnology vol. 6, Rehm et al., eds. VCH: Weinheim, and references contained therein), lipids, both saturated and polyunsaturated fatty acids (e.g., arachidonic acid), diols (e.g., propane diol, and butane diol), carbohydrates (e.g., hyaluronic acid and trehalose), aromatic compounds (e.g., aromatic amines, vanillin, and indigo), vitamins and cofactors (as described in Ullmann's Encyclopedia of Industrial Chemistry, vol. A27, Vitamins, p. 443-613 (1996) VCH: Weinheim and references therein; and Ong, A. S., Niki, E. & Packer, L. (1995) Nutrition, Lipids, Health, and Disease” Proceedings of the UNESCO/Confederation of Scientific and Technological Associations in Malaysia, and the Society for Free Radical Research, Asia, held Sept. 1-3, 1994 at Penang, Malaysia, AOCS Press, (1995)), enzymes, and all other chemicals described in Gutcho (1983) Chemicals by Fermentation, Noyes Data Corporation, ISBN: 0818805086 and references therein. The metabolism and uses of certain of these fine chemicals are further explicated below.

### Tocopherol and Carotenoid Metabolism and Uses

Vitamins, cofactors, and nutraceuticals comprise another group of fine chemical molecules which higher animals have lost the ability to synthesize and so must ingest. These molecules are readily synthesized by other organisms, such as bacteria, fungi, algae and plants. These molecules are either bioactive substances themselves, or are precursors of biologically active substances which may serve as electron carriers or intermediates in a variety of metabolic pathways. Besides their nutritive value, these compounds also have significant industrial value as coloring agents, antioxidants, and catalysts or other processing aids. (For an overview of the structure, activity, and industrial applications of these compounds, see, for example, Ullman's Encyclopedia of Industrial Chemistry, “Vitamins” vol. A27, p. 443-613, VCH: Weinheim, 1996.) The term “vitamin” is art-recognized, and includes nutrients which are required by an organism for normal functioning, but which that organism cannot synthesize by itself. One preferred embodiment of the instant invention pertains to vitamin E compounds (tocopherols) and their production in plants. The group of vitamins may encompass cofactors and nutraceutical compounds. The language “cofactor” includes nonproteinaceous compounds required for a normal enzymatic activity to occur. Such compounds may be organic or inorganic; the cofactor molecules of the invention are preferably organic. The term “nutraceutical” includes dietary supplements having health benefits in plants and animals, particularly humans. Examples of such molecules are vitamins, antioxidants, and also certain lipids (e.g., polyunsaturated fatty acids).

The biosynthesis of these molecules in organisms capable of producing them, such as bacteria and plants, has been largely characterized (Friedrich, W. “Handbuch der Vitamine”, Urban und Schwarzenberg, 1987; Ullman's Encyclopedia of Industrial Chemistry, “Vitamins” vol. A27, p. 443-613, VCH: Weinheim, 1996; Michal, G. (1999) Biochemical Pathways: An Atlas of Biochemistry and Molecular Biology, John Wiley & Sons; Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sept. 1-3, 1994 at Penang, Malaysia, AOCS Press: Champaign, Ill. X, 374 S).

The metabolism and uses of certain of these vitamins are further explicated below.

Tocopherols (Vitamin E):

The fat-soluble vitamin E has received great attention for its essential role as an antioxidant in nutritional and clinical applications (Liebler DC 1993. Critical Reviews in Toxicology 23(2):147-169) thus representing a good area for food design, feed applications and pharmaceutical applications. In addition, benefitial effects are encountered in retarding diabetes-related high-age damages, anticancerogenic effects as well as a protective role against erythreme and skin aging. Alpha-tocopherol as the most important antioxidans helps to prevent the oxidation of unsatturated fatty acids by oxygen in humans by its redox potential (Erin A N, Skrypin V V, Kragan V E 1985, Biochim. Biophy. Acta 815: 209).

The demand for this vitamin has increased year after year. The supply of tocopherols has been limited to the chemically synthesized racemate of alpha-tocopherol or a mixture of alpha-, beta(gamma)- and delta-tocopherols from vegetable oils. Altogether, the group of compounds with vitamin E activity now comprises alpha-, beta-, gamma-, and delta-tocopherol as well as alpha-, beta-, gamma-, and delta-tocotrienol.

Biologically, tocopherols are indispensable components of the lipid bilayer of cell membranes. A reduction of availability of tocopheroles leads to structural and functional damaging of membranes. This stabilizing effect of the tocopherols on membranes is accepted to be related to three functions: 1) tocopherols react with lipid peroxide radicals, 2) quenching of reactive molecular oxygen, and 3) reducing the molecular mobility of the membrane bilayer by the formation of tocopherol-fatty acids complexes.

In addition to the occurrence of tocopherols in plants, their presence has been determined in various microorganisms, especially in many chlorophyll-containing organisms (Taketomi H, Soda K, Katsui G 1983, Vitamins (Japan) 57: 133-138). Algae, for example Euglenia gracilis, also contain tocopherols and Euglenia gracilis is described as a suitable host for the production of tocopherols since the most valuable form alpha-tocopherol is the major component of tocopherols (Shigeoka S, Onishi T, Nakano Y, Kitaoka S 1986, Agric. Biol. Chem. 50: 1063-1065). Also, yeasts and bacteria were found to synthesize tocopherols (Forbes M, Zilliken F, Roberts G, György P 1958, J. Am. Chem. Soc. 80: 385-389; Hughes and Tove 1982, J Bacteriol., 151: 1397-1402; Ruggeri B A, Gray R J H, Watkins T R, Tomlins R I 1985, Appl. Env. Microbiol. 50:1404-1408).

Tocopherol is synthesized from geranylgeranylpyrophosphate which is generated from isopentenylpyrophosphate (IPP). IPP can be produced via two independent pathways. One pathway is located in the cytoplasm, whereas the other is located in the chloroplasts (for descriptions and reviews see Trelfall D R, Whistance G R in Aspects of Terpenoid Chemistry and Biochemistry, Goodwin T W Ed., Academic Press, London, 1971: 357-404; Michal G Ed. 1999, Biochemical Pathways, Spektrum Akademischer Verlag GmbH Heidelberg, and references cited therein; McCaskill D, Croteau R 1998, Tibtech 16: 349-355 and references cited therein; Rhomer M 1998, Progress in Drug Research 50: 135-154; Lichtenthaler H K 19998, Annu. Rev. Plant Physiol. Plant Mol. Biol. 50: 47-65; Lichtenthaler H K, Schwender J, Disch A, Rhomer M 1997, FEBS Letters 400: 271-274; Schultz G, Soll J 1980 Deutsche Tierärzliche Wochenschrift 87: 401-424; Arigoni D, Sagner S, Latzel C, Eisenreich W, Bacher A, Zenk, M H 1997 Proc. Natl. Acad. Sci. USA 94(2): 10600-10605). For a general review of isoprene biosynthesis and products derived from that pathway (Chappell 1995, Annu. Rev. Plant Physiol. Plant Mol. Biol. 46:521-547; Sharkey T D, 1996, Endeavor 20: 74-78).

The cyclic structures which are required for tocopherol biosynthesis are quinones. Quinones are synthesized from products of the shikimate pathway (for review see Dewick P M 1995, Natural Products Reports 12(6): 579-607; Weaver L M, Herrmann K M 1997, Trends in Plant Science 2(9): 346-351; Schmid J, Amrhein N 1995, Phytochemistry 39(4): 737-749).

Plant genes originating from Physcomitrella patens can be used to modify tocopherol metabolism in plants as well as algae and microorganisms enabling these host cells to increase their capacity to produce tocopherols as well as improving survival and fitness of the host cell. Thereby, one or several genes, alone or in combination, preferably of the genes encoding the γ-tocopherol-methyltransferase (gamma-TMT type I), 2-methyl-6-phytylplastoquinol methyltransferase (garnma-TMT type II) or 4-hydroxyphenylpyruvate dioxygenase, can be used to modify the tocopherol metabolism.

Carotenoids:

Carotenoids are naturally occurring pigments synthesized as hydrocarbons (carotenes) and their oxygenated derivatives (xantophylls) are produced by plants and microorganisms. The application potential was broadly investigated during the last 20 years. Besides the use of carotenoids as coloring agents, it is assumed that carotenoids play an important role in the prevention of cancer (Rice-Evans et al. 1997, Free Radic. Res. 26:381-398; Gerster 1993, Int. J. Vitam. Nutr. Res. 63:93-121; Bendich 1993, Ann. New York Acad. Sci. 691:61-67) thus representing a good area for food design, feed applications and pharmaceutical applications.

The major function of carotenoids in plants and microoganisms is in protection against oxidative damage by quenching photosensensitizers interacting with singlet oxygen and scavenging peroxiradicals, thus preventing the accumulation of harmful oxygen species and subsequent maintainance of membrane integrity (Havaux 1998, Trends in Plant Science Vol 3 (4):147-151; Krinsky 1994, Pur Appl. Chem. 66:1003-1010). Thus an application is also given for the optimization of fermentation processes with respect to lesser susceptibility to oxidative damage. For a review of biotechnological potential see Sandmann et al. (1999, Tibtech 17; 233-237).

Plant genes originating from Physcomitrella patens can be used to modify carotenoid metabolism in plants as well as algae and microorganisms enabling these host cells to increase their capacity to produce carotenoids and to produce newly designed carotenoids as well as improving survival and fitness of the host cell due to the expression of plant acrotenoid biosynthetic genes.

Due to results obtained in labelling experiments it is clear that carotenes arise from the isoprenoid biosynthesis pathway via geranylgeranylpyrophosphate synthesis. For review of products of the isoprenoid biosynthetic pathway including carotenoids see Chappell 1995, Annu. Rev. Plant Physiol. Plant Mol. Biol. 46:521-547. The biosynthesis of carotenoids in microorganims and plants is described in following articles and references therein: Armstrong 1997, Annu. Rev. Microbiol., 51:629-659; Sandmannn 1994, Eur. J. Biochem. 223:7-24; Misawa et al. 1995, J. Bacteriol. 177 (22):6575-6584; -Hirschberg et al. 1997, Pure & Appl. Chem 69 (10):2151-2158; Lotan & Hirschberg 1995, FEBS Letters 364:125-128; U.S. Pat. No. 5,916,791).

The large-scale production of the fine chemical compounds described above has largely relied on cell-free chemical syntheses. Production through large scale fermentation of microorganism has not yet proven to be useful, due to unsufficient efficience and high costs. Allthough not yet applicable for large scale production it has been shown that production of fine chemicals can be enhanced in genetically modified plants as exemplified for phytoene in rice (Burkhardt et al. Plant Journal 11(5):1071-8, 1997) and vitamin E in Arabidopsis thaliana and other plants (Shintani and DellaPenna. Science 282(5396):2098-100, 1998; W099/23231). Increased amounts of such compounds in plants are especially appreciable because the plants can be directly applied for food and feed purposes.

### Elements and Methods of the Invention

The present invention is based, at least in part, on the discovery of novel molecules, referred to herein as TCMRP nucleic acid and protein molecules, which play a role in or function in one or more cellular metabolic pathways in Physcomitrella patens. In one embodiment, the TCMRP molecules catalyze an enzymatic reaction involving one or more tocopherol and/or carotinoid metabolic pathways. In a preferred embodiment, the activity of the TCMRP molecules of the present invention in one or more Physcomitrella patens metabolic pathways for tocopherols and carotenoids has an impact on the production of a desired fine chemical by this organism. In a particularly preferred embodiment, the TCMRPs encoded by TCMRP nucleotides of the invention are modulated in activity, such that the mircroorganisms' or plants' metabolic pathways which the TCMRPs of the invention regulate are modulated in yield, production, and/or efficiency of production and/or transport of a desired fine chemical by microorganisms and plants.

The language, TCMRP or TCMRP polypeptide includes proteins which play a role in, e.g., catalyze an enzymatic reaction, in one or more tocopherol and carotenoid metabolic pathways in microorganisms and plants. Examples of TCMRPs include those encoded by the TCMRP genes set forth in Table 1 and Appendix A. The terms TCMRP gene or TCMRP nucleic acid sequence include nucleic acid sequences encoding an TCMRP, which consist of a coding region or a part thereof and/or also corresponding untranslated 5′ and 3′ sequence regions. Examples of TCMRP genes include those set forth in Table 1. The terms production or productivity are art-recognized and include the concentration of the fermentation product (for example, the desired fine chemical) formed within a given time and a given fermentation volume (e.g., kg product per hour per liter). The term efficiency of production includes the time required for a particular level of production to be achieved (for example, how long it takes for the cell to attain a particular rate of output of a fine chemical). The term yield or product/carbon yield is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms biosynthesis or a biosynthetic pathway are art-recognized and include the synthesis of a compound, preferably an organic compound, by a cell from intermediate compounds in what may be a multistep and highly regulated process. The terms degradation or a degradation pathway are art-recognized and include the breakdown of a compound, preferably an organic compound, by a cell to degradation products (generally speaking, smaller or less complex molecules) in what may be a multistep and highly regulated process. The language metabolism is art-recognized and includes the totality of the biochemical reactions that take place in an organism. The metabolism of a particular compound, then, (e.g., the metabolism of a fatty acid) comprises the overall biosynthetic, modification, and degradation pathways in the cell related to this compound.

In another embodiment, the TCMRP molecules of the invention are capable of modulating the production of a desired molecule, such as a fine chemical, in microorganisms and plants. There are a number of mechanisms by which the alteration of an TCMRP of the invention may directly affect the yield, production, and/or efficiency of production of a fine chemical from a microorganisms or plant strain incorporating such an altered protein. Those TCMRPs involved in the transport of fine chemical molecules within or from the cell may be increased in number or activity such that greater quantities of these compounds are transported across membranes. Similarly, those TCMRPs involved in the import of nutrients necessary for the biosynthesis of one or more fine chemicals may be increased in number or activity such that these precursor, cofactor, or intermediate compounds are increased in concentration within a desired cell. Further TCMRPs may be increased in number or activity which lead to a regeneration of a pool of fine chemicals in a desired state. The mutagenesis of one or more TCMRP genes of the invention may also result in TCMRPs having altered activities which indirectly impact the production of one or more desired fine chemicals from microorganisms, algae and plants. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall yield or rate of production of one of these desired fine chemicals may be increased.

It is also possible that such alterations in the protein and nucleotide molecules of the invention may improve the production of other fine chemicals besides the tocopherols and carotenoids. Metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the proteins of the invention, the production or efficiency of activity of another fine chemical biosynthetic or degradative pathway may be impacted. For example, amino acids serve as the structural units of all proteins, yet may be present intracellularly in levels which are limiting for protein synthesis; therefore, by increasing the efficiency of production or the yields of one or more amino acids within the cell, proteins, such as biosynthetic or degradative proteins, may be more readily synthesized. Likewise, an alteration in a metabolic pathway enzyme such that a particular side reaction becomes more or less favored may result in the over- or under-production of one or more compounds which are utilized as intermediates or substrates for the production of a desired fine chemical.

TCMRPs of the invention involved in the export of waste products may be increased in number or activity such that the normal metabolic wastes of the cell (possibly increased in quantity due to the overproduction of the desired fine chemical) are efficiently exported before they are able to damage nucleotides and proteins within the cell (which would decrease the viability of the cell) or to interfere with fine chemical biosynthetic pathways (which would decrease the yield, production, or efficiency of production of the desired fine chemical). Further, the relatively large intracellular quantities of the desired fine chemical may in itself be toxic to the cell, so by increasing the activity or number of transporters able to export this compound from the cell, one may increase the viability of the cell in culture, in turn leading to a greater number of cells in the culture producing the desired fine chemical.

The TCMRPs of the invention may also be manipulated such that the relative amounts of different tocopherols and carotenoids are produced. The isolated nucleic acid sequences of the invention are contained within the genome of a Physcomitrella patens strain available through the moss collection of the University of Hamburg. The nucleotide sequence of the isolated Physcomitrella patens TCMRP cDNAs and the predicted amino acid sequences of the respective Physcomitrella patens TCMRPs are shown in Appendices A and B, respectively.

Computational analyses were performed which classified and/or identified these nucleotide sequences as sequences which encode proteins involved in the metabolism of amino acids, vitamins, cofactors, nutraceuticals, nucleotide or nucleosides.

The present invention also pertains to proteins which have an amino acid sequence which is substantially homologous to an amino acid sequence of Appendix B. As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

The TCMRP or a biologically active portion or fragment thereof of the invention can catalyze an enzymatic reaction in one or more tocopherol and carotenoid metabolic pathways in plants and microorganisms, or have one or more of the activities set forth in Table 1. Various aspects of the invention are described in further detail in the following subsections:

### A. Isolated Nucleic Acid Molecules

One aspect of the invention pertains to isolated nucleic acid molecules that encode TCMRP polypeptides or biologically active portions thereof, as well as nucleic acid fragments sufficient for use as hybridization probes or primers for the identification or amplification of TCMRP-encoding nucleic acid (e.g., TCMRP DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated TCMRP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, a Physcomitrella patens cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, or a portion thereof, can be isolated using standard molecular biology techniques and the sequence information provided herein. For example, a P. patens TCMRP cDNA can be isolated from a P. patens library using all or portion of one of the sequences of Appendix A as a hybridization probe and standard hybridization techniques (e.g., as described in Sambrook et al., Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, MRNA can be isolated from plant cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an TCMRP nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A. The sequences of Appendix A correspond to the Physcomitrella patens TCMRP cDNAs of the invention. This cDNA comprises sequences encoding TCMRPs (i.e., the “coding region”, indicated in each sequence in Appendix A), as well as 5′ untranslated sequences and 3′ untranslated sequences. Alternatively, the nucleic acid molecule can comprise only the coding region of any of the sequences in Appendix A or can contain whole genomic fragments isolated from genomic DNA. In another embodiment, the sequences of Appendix A can have corresponding longest nucleic acid molecules, e.g. full length or nearly full length nucleic acid molecules encoding a TCMRP. The corresponding clone name is given in Table 1.

For the purposes of this application, it will be understood that each of the sequences set forth in Appendix A has an identifying entry number. Each of these sequences comprises up to three parts: a 5′ upstream region, a coding region, and a downstream region. Each of these three regions is identified by the same entry number designation to eliminate confusion. The recitation one of the sequences in Appendix A, then, refers to any of the sequences in Appendix A, which may be distinguished by their differing entry number designations. The coding region of each of these sequences is translated into a corresponding amino acid sequence, which is set forth in Appendix B. The sequences of Appendix B are identified by the same entry numbers designations as Appendix A, such that they can be readily correlated. For example, the amino acid sequence in Appendix B designated 41_bd10_g03rev is a translation of the coding region of the nucleotide sequence of nucleic acid molecule 41_bd10_g03rev in Appendix A, and the amino acid sequence in Appendix B designated 68_ck12_d10fwd is a translation of the coding region of the nucleotide sequence of nucleic acid molecule 68_ck12_d10fwd in Appendix A.

In another preferred embodiment, an isolated nucleic acid molecule of the invention comprises a nucleic acid molecule which is a complement of one of the nucleotide sequences shown in Appendix A, or a portion thereof. A nucleic acid molecule which is complementary to one of the nucleotide sequences shown in Appendix A is one which is sufficiently complementary to one of the nucleotide sequences shown in Appendix A such that it can hybridize to one of the nucleotide sequences shown in Appendix A, thereby forming a stable duplex.

In still another preferred embodiment, an isolated nucleic acid molecule of the invention comprises a nucleotide sequence which is at least about 50-60%, preferably at least about 60-70%, more preferably at least about 70-80%, 80-90%, or 90-95%, and even more preferably at least about 95%, 96%, 97%, 98%, 99% or more homologous to a nucleotide sequence shown in Appendix A, or a portion thereof. In an additional preferred embodiment, an isolated nucleic acid molecule of the invention comprises a nucleotide sequence which hybridizes, e.g., hybridizes under stringent conditions, to one of the nucleotide sequences shown in Appendix A, or a portion thereof.

Moreover, the nucleic acid molecule of the invention can comprise only a portion of the coding region of one of the sequences in Appendix A, for example a fragment which can be used as a probe or primer or a fragment encoding a biologically active portion of an TCMRP. The nucleotide sequences determined from the cloning of the TCMRP genes from P. patens allows for the generation of probes and primers designed for use in identifying and/or cloning TCMRPhomologues in other cell types and organisms, as well as TCMRP homologues from other mosses or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone TCMRP homologues. Probes based on the TCMRP nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme cofactor. Such probes can be used as a part of a genomic marker test kit for identifying cells which misexpress an TCMRP, such as by measuring a level of an TCMRP-encoding nucleic acid in a sample of cells, e.g., detecting TCMRP mRNA levels or determining whether a genomic TCMRPgene has been mutated or deleted.

In one embodiment, the nucleic acid molecule of the invention encodes a protein or portion thereof which includes an amino acid sequence which is sufficiently homologous to an amino acid sequence of Appendix B such that the protein or portion thereof maintains the ability to catalyze an enzymatic reaction in a tocopherol or carotenoid metabolic pathway in microorganisms or plants. As used herein, the language “sufficiently homologous” refers to proteins or portions thereof which have amino acid sequences which include a minimum number of identical or equivalent (e.g., an amino acid residue which has a similar side chain as an amino acid residue in one of the sequences of Appendix B) amino acid residues to an amino acid sequence of Appendix B such that the protein or portion thereof is able to catalyze an enzymatic reaction in a tocopherol or carotenoid metabolic pathway in microorganisms or plants. Protein members of such metabolic pathways, as described herein, function to catalyze the biosynthesis or degradation or stabilisation of one or more tocopherols or carotenoids. Examples of such activities are also described herein. Thus, the function of an TCMRP” contributes either directly or indirectly to the yield, production, and/or efficiency of production of one or more fine chemicals. Examples of TCMRP activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of Appendix B.

Portions of proteins encoded by the TCMRP nucleic acid molecules of the invention are preferably biologically active portions of one of the TCMRP. As used herein, the term “biologically active portion of an TCMRP” is intended to include a portion, e.g., a domain/motif, of an TCMRP that participates in the metabolism of fine chemicals like amino acids, vitamins, cofactors, nutraceuticals, nucleotides, or nucleosides in microorganisms or plants or has an activity as set forth in Table 1. To determine whether an TCMRP or a biologically active portion thereof can participate in the metabolism of fine chemicals like amino acids, vitamins, cofactors, nutraceuticals, nucleotides, or nucleosides in microorganisms or plants, an assay of enzymatic activity may be performed. Such assay methods are well known to those skilled in the art, as detailed in Example 17 of the Exemplification.

Additional nucleic acid fragments encoding biologically active portions of an TCMRP can be prepared by isolating a portion of one of the sequences in Appendix B, expressing the encoded portion of the TCMRP or peptide (e.g., by recombinant expression in vitro) and assessing the activity of the encoded portion of the TCMRP or peptide.

The invention further encompasses nucleic acid molecules that differ from one of the nucleotide sequences shown in Appendix A (and portions thereof) due to degeneracy of the genetic code and thus encode the same TCMRP as that encoded by the nucleotide sequences shown in Appendix A. In another embodiment, an isolated nucleic acid molecule of the invention has a nucleotide sequence encoding a protein having an amino acid sequence shown in Appendix B. In a still further embodiment, the nucleic acid molecule of the invention encodes a full length Physcomitrella patens protein which is substantially homologous to an amino acid sequence of Appendix B (encoded by an open reading frame shown in Appendix A).

In addition to the Physcomitrella patens TCMRP nucleo tide sequences shown in Appendix A, it will be appreciated by those skilled in the art that DNA sequence polymorphisms that lead to changes in the amino acid sequences of TCMRPs may exist within a population (e.g., the Physcomitrella patens population). Such genetic polymorphism in the TCMRP gene may exist among individuals within a population due to natural variation. As used herein, the terms “gene” and “recombinant gene” refer to nucleic acid molecules comprising an open reading frame encoding an TCMRP, preferably a Physcomitrella patens TCMRP. Such natural variations can typically result in 1-5% variance in the nucleotide sequence of the TCMRP gene. Any and all such nucleotide variations and resulting amino acid polymorphisms in TCMRPsthat are the result of natural variation and that do not alter the functional activity of TCMRPs are intended to be within the scope of the invention.

Nucleic acid molecules corresponding to natural variants and non-Physcomitrella patens homologues of the Physcomitrella patens TCMRP cDNA of the invention can be isolated based on their homology to Physcomitrella patens TCMRP nucleic acid disclosed herein using the Physcomitrella patens cDNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural Physcomitrella patens TCMRP.

In addition to naturally-occurring variants of the TCMRPsequence that may exist in the population, the skilled artisan will further appreciate that changes can be introduced by mutation into a nucleotide sequence of Appendix A, thereby leading to changes in the amino acid sequence of the encoded TCMRP, without altering the functional ability of the TCMRP. For example, nucleotide substitutions leading to amino acid substitutions at “non-essential” amino acid residues can be made in a sequence of Appendix A. A “non-essential” amino acid residue is a residue that can be altered from the wild-type sequence of one of the TCMRP proteins (Appendix B) without altering the activity of said TCMRP, whereas an “essential” amino acid residue is required for TCMRP activity. Other amino acid residues, however, (e.g., those that are not conserved or only semi-conserved in the domain having TCMRP activity) may not be essential for activity and thus are likely to be amenable to alteration without altering TCMRP activity.

Accordingly, another aspect of the invention pertains to nucleic acid molecules encoding TCMRPs that contain changes in amino acid residues that are not essential for TCMRP activity. Such TCMRPs differ in amino acid sequence from a sequence contained in Appendix B yet retain at least one of the TCMRP activities described herein. In one embodiment, the isolated nucleic acid molecule comprises a nucleotide sequence encoding a protein, wherein the protein comprises an amino acid sequence at least about 50% homologous to an amino acid sequence of Appendix B and is able to catalyze an enzymatic reaction in a tocopherol or carotenoid metabolic pathway in P. patens, or has one or more activities set forth in Table 1. Preferably, the protein encoded by the nucleic acid molecule is at least about 50-60% homologous to one of the sequences in Appendix B, more preferably at least about 60-70% homologous to one of the sequences in Appendix B, even more preferably at least about 70-80%, 80-90%, 90-95% homologous to one of the sequences in Appendix B, and most preferably at least about 96%, 97%, 98%, or 99% homologous to one of the sequences in Appendix B.

To determine the percent homology of two amino acid sequences (e.g., one of the sequences of Appendix B and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B) is occupied by the same amino acid residue or nucleotide as the corresponding position in the other sequence (e.g., a mutant form of the sequence selected from Appendix B), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=numbers of identical positions/total numbers of positions×100).

An isolated nucleic acid molecule encoding an TCMRP homologous to a protein sequence of Appendix B can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an TCMRP is preferably replaced with another amino acid residue from the same side chain family. Alternatively, in another embodiment, mutations can be introduced randomly along all or part of an TCMRP coding sequence, such as by saturation mutagenesis, and the resultant mutants can be screened for an TCMRP activity described herein to identify mutants that retain TCMRP activity. Following mutagenesis of one of the sequences of Appendix A, the encoded protein can be expressed recombinantly and the activity of the protein can be determined using, for example, assays described herein (see Example 17 of the Exemplification).

In addition to the nucleic acid molecules encoding TCMRPs described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TCMRP cDNA coding strand, or to only a portion thereof. In one embodiment, an antisense nucleic acid molecule is antisense to a “coding region” of the coding strand of a nucleotide sequence encoding an TCMRP. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding TCMRPs. The term “noncoding region” refers to 5′ and 3′ sequences which flank the coding region that are not translated into amino acids (i.e., also referred to as 5′ and 3′ untranslated regions).

Given the coding strand sequences encoding TCMRPs disclosed herein (e.g., the sequences set forth in Appendix A), antisense nucleic acids of the invention can be designed according to the rules of Watson and Crick base pairing. The antisense nucleic acid molecule can be complementary to the entire coding region of TCMRP cDNA, but more preferably is an oligonucleotide which is antisense to only a portion of the coding or noncoding region of TCMRP mRNA. For example, the antisense oligonucleotide can be complementary to the region surrounding the translation start site of TCMRP MRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, -7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular MRNA and/or genomic DNA encoding an TCMRP to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic including plant promoters are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987)Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave TCMRP mRNA transcripts to thereby inhibit translation of TCMRP mRNA. A ribozyme having specificity for an TCMRP-encoding nucleic acid can be designed based upon the nucleotide sequence of an TCMRP cDNA disclosed herein. For example, a derivative of a Tetrahymena L-19 IVS RNA can be constructed in which the nucleotide sequence of the active site is complementary to the nucleotide sequence to be cleaved in an TCMRP -encoding mRNA. See, e.g., Cech et al. U.S. Pat. No. 4,987,071 and Cech et al. U.S. Pat. No. 5,116,742. Alternatively, TCMRP mRNA can be used to select a catalytic RNA having a specific ribonuclease activity from a pool of RNA molecules. See, e.g., Bartel, D. and Szostak, J. W. (1993) Science 261:1411-1418.

Alternatively, TCMRP gene expression can be inhibited by targeting nucleotide sequences complementary to the regulatory region of an TCMRP nucleotide sequence (e.g., an TCMRP promoter and/or enhancers) to form triple helical structures that prevent transcription of an TCMRP gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

### B. Recombinant Expression Vectors and Host Cells

Another aspect of the invention pertains to vectors, preferably expression vectors, containing a nucleic acid encoding an TCMRP (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed.

Suitable vectors for plants are described, inter alia, in “Methods in Plant Molecular Biology and Biotechnology” (CRC Press), chapter 6/7, pp. 71-119 (1993).

Within a recombinant expression vector, “operably linked” is intended to mean that the nucleotide sequence of interest is linked to the regulatory sequence(s) in a manner which allows for expression of the nucleotide sequence are fused to each other so that both sequences fulfil the proposed function addicted to the sequence used. (e.g., in an in vitro transcription/ translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990) or in Gruber and Crosby, in: Methods in Plant Molecular Biology and Biotechnolgy, CRC Press, Boca Raton, Fla., eds.: Glick and Thompson, Chapter 7, 89-108 including the references therein.

Other advantageous regulatory sequences are present in, for example, the Gram-positive promoters amy and SPO2, in the yeast or fungal promoters ADC1, MFa, AC, P-60, CYC1, GAPDH, TEF, rp28, ADH or in the plant promoters CaMV/35S [Franck et al., Cell 21(1980) 285-294], PRP1 [Ward et al., Plant. Mol. Biol. 22 (1993)], SSU, OCS, leb4, usp, STLS1, B33, nos or in the ubiquitin or phaseolin promoters.

As regards plants as genetically modified organisms, any promoter capable of governing the expression of foreign genes in plants is suitable in principle as promoter of the expression cassette.

Preferably, it is in particular a plant promoter or a promoter derived from a plant virus which is used. Particularly preferred is the cauliflower mosaic virus CaMV 35S promoter (Franck et al., Cell 21 (1980), 285-294). As is known, this promoter comprises various recognition sequences for transcriptional effectors which, in totality, lead to permanent and constitutive expression of the gene which has been inserted (Benfey et al., EMBO J. 8 (1989), 2195-2202).

The expression cassette can also comprise a pathogen-inducible or chemically inducible promoter by means of which expression of the exogenous TCMRP genes in the plant can be governed at a particular point in time.

Examples of such promoters which can be used are, for example, the PRP1 promoter (Ward et al., Plant. Mol. Biol. 22 (1993), 361-366), a salicylic-acid-inducible promoter (WO95/19443), a benzenesulfonamide-inducible promoter (EP-A 388186), a tetracyclin-inducible promoter (Gatz et al., (1992) Plant J. 2, 397-404), an abscisic-acid-inducible promoter (EP-A 335528) or an ethanol- or cyclohexanone-inducible promoter (WO 93/21334).

Furthermore, preferred promoters are in particular those which ensure expression in tissues or plant organs in which, for example, the biosynthesis of tocopherol or its precursors takes place or in which the products are advantageously accumulated.

Promoters which must be mentioned in particular are those for the entire plant owing to constitutive expression, such as, for example, the CaMV promoter, the Agrobacterium OCS promoter (octopine synthase), the Agrobacterium NOS promoter (nopaline synthase), the ubiquitin promoter, promoters of vacuolar ATPase subunits, or the promoter of a proline-rich protein from wheat (wheat WO 9113991).

Furthermore, promoters which must be mentioned in particular are those which ensure leaf-specific expression. Promoters which must be mentioned are the potato cytosolic FBPase promoter (WO9705900), the Rubisco (ribulose-1,5-bisphosphate carboxylase) SSU (small subunit) promoter or the potato ST-LSI promoter (Stockhaus et al., EMBO J. 8 (1989), 2445-245).

### Examples of Further Suitable Promoters Are

specific promoters for tubers, storage roots or roots such as, for example, the patatin promoter class I (B33), the potato cathepsin D inhibitor promoter, the starch synthase (GBSS1) promoter or the sporamin promoter, fruit-specific promoters such as, for example, the tomato fruit-specific promoter (EP 409625), fruit-maturation-specific promoters such as, for example, the tomato fruit-maturation-specific promoter (WO 9421794), flower-specific promoters such as, for example, the phytoene synthase promoter (WO 9216635) or the promoter of the P-rr gene (WO 9822593) or specific plastid or chromoplast promoters such as, for example, the RNA polymerase promoter (WO 9706250).

Other promoters which can advantageously be used are the Glycine max phosphoribosyl pyrophosphate amidotransferase promoter (see also Genbank Accession Number U87999) or another nodia-specific promoter as described in EP 249676.

In principle, all natural promoters together with their regulatory sequences like those mentioned above can be used for the process according to the invention. In addition, synthetic promoters can also be used advantageously.

Further, a seed-specific promoter (preferably the phaseolin promoter (U.S. Pat. No. 5,504,200), the USP promoter (Baumlein, H. et al., Mol. Gen. Genet. (1991) 225 (3), 459-467), the Brassica Bce4 gene promoter (WO 9113980) or the LEB4 promoter (Fiedler and Conrad, 1995)), are advantagous.

Regulatory sequences include those which direct constitutive expression of a nucleotide sequence in many types of host cell and those which direct expression of the nucleotide sequence only in certain host cells or under certain conditions. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., TCMRPs, mutant forms of TCMRPs, fusion proteins, etc.).

The recombinant expression vectors of the invention can be designed for expression of TCMRPs in prokaryotic or eukaryotic cells. For example, TCMRP genes can be expressed in bacterial cells such as C. glutamicum, insect cells (using baculovirus expression vectors), yeast and other fungal cells (see Romanos, M.A. et al. (1992) Foreign gene expression in yeast: a review, Yeast 8: 423-488; van den Hondel, C. A. M. J. J. et al. (1991) Heterologous gene expression in filamentous fungi, in: More Gene Manipulations in Fungi, J. W. Bennet & L. L. Lasure, eds., p. 396-428: Academic Press: San Diego; and van den Hondel, C. A. M. J. J. & Punt, P. J. (1991) Gene transfer systems and vector development for filamentous fungi, in: Applied Molecular Genetics of Fungi, Peberdy, J. F. et al., eds., p. 1-28, Cambridge University Press: Cambridge), algae (Falciatore et al., 1999, Marine Biotechnology. 1 (3):239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in WO9801572 and multicellular plant cells (see Schmidt, R. and Willmitzer, L. (1988), High efficiency Agrobacterium tumefaciens-mediated transformation of Arabidopsis thaliana leaf and cotyledon explants, Plant Cell Rep.: 583-586); Plant Molecular Biology and Biotechnology, C Press, Boca Raton, Fla., chapter 6/7, S.71-119 (1993); F. F. White, B. Jenes et al., Techniques for Gene Transfer, in: Transgenic Plants, Vol. 1, Engineering and Utilization, eds.: Kung und R. Wu, Academic Press (1993), 128-43; Potrykus, Annu. Rev. Plant Physiol. Plant Molec. Biol. 42 (1991), 205-225; or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the TCMRP is cloned into a pGEX expression vector to create a vector encoding a fusion protein comprising, from the N-terminus to the C-terminus, GST-thrombin cleavage site-X protein. The fusion protein can be purified by affmity chromatography using glutathione-agarose resin. Recombinant TCMRP unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 romoter.

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20:2111-2118). Such alteration of nucleic acid sequences of the invention can be carried out by standard DNA synthesis techniques.

In another embodiment, the TCMRP expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) ”Gene transfer systems and vector development for filamentous fungi, in: Applied Molecular Genetics of Fungi, J. F. Peberdy, et al., eds., p. 1-28, Cambridge University Press: Cambridge.

Alternatively, the TCMRPs of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and -eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the fetoprotein promoter (Campes and Tilghman (1989) Genes Dev. 3:537-546).

In another embodiment, the TCMRPs of the invention may be expressed in unicellular plant cells (such as algae) see Falciatore et al., 1999, Marine Biotechnology.1 (3):239-251 and references therein and plant cells from higher plants (e.g., the spermatophytes, such as crop plants). Examples of plant expression vectors include those detailed in: Becker, D, Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation, Nucl. Acid. Res. 12: 8711-8721; Vectors for Gene Transfer in Higher Plants; in: Transgenic Plants, Vol. 1, Engineering and Utilization, eds.: Kung und R. Wu, Academic Press, 1993, S. 15-38.

Further, TCMRP genes can be incorporated into a derivative of the transformation vector pBin-19 with 35S promoter (Bevan, M., Nucleic Acids Research 12: 8711-8721 (1984)).

A plant expression cassette preferably contains regulatory sequences capable to drive gene expression in plants cells and which are operably linked so that each sequence can fulfil its function such as termination of transcription such as polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3 (1984), 835 ff) or functional equivalents therof but also all other terminators are suitable.

As plant gene expression is very often not limited on transcriptional levels a plant expression cassette preferably contains other operably linked sequences like translational enhancers such as the overdrive-sequence containing the 5′-untranlated leader sequence from tobacco mosaic virus enhancing the protein per RNA ratio (Gallie et al 1987, Nucl. Acids Research 15:8693-8711).

Plant gene expression has to be operably linked to an appropriate promoter conferring gene expression in a timely , cell or tissue specific manner. Preferrred are promoters driving constitutitive expression (Benfey et al., EMBO J. 8 (1989) 2195-2202) like those derived from plant viruses like the 35S CAMV (Franck et al., Cell 21(1980) 285-294), the 19S CaMV (see also U.S. Pat. No. 5352605 and WO8402913) or plant promoters like those from Rubisco small subunit described in U.S. Pat. No. 4,962,028. WO 8705629, WO 9204449.

Other preferred sequences for use operable linkage in plant gene expression cassettes are targeting-sequences necessary to direct the gene-product in its appropriate cell compartment (for review see Kermode, Crit. Rev. Plant Sci. 15, 4 (1996), 285-423 and references cited therin) such as the vacuole, the nucleus, all types of plastids like amyloplasts, chloroplasts, chromoplasts, the extracellular space, mitochondria, the endoplasmic reticulum, oil bodies, peroxisomes and other compartments of plant cells.

It is also possible to use expression cassettes whose DNA sequence encodes, for example, a fusion protein, part of the fusion protein being a transit peptide which governs the translocation of the polypeptide. Preferred are chloroplast-specific transit peptides, which are cleaved enzymatically from the moiety after the TCMRP gene product has been translocated into the chloroplasts. Particularly preferred is the transit peptide which is derived from the plastid Nicotiana tabacum transketolase or from another transit peptide (for example the Rubisco small subunit transit peptide, or the ferredoxin NADP oxidoreductase and also the isopentenyl pyrophosphate isomerase-2) or its functional equivalent.

Especially preferred are DNA sequences of three cassettes of the plastid transit peptide of the tobacco plastid transketolase in three reading frames as KpnI/BamHI fragments with an ATG codon in the NcoI cleavage site:

pTPO9
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGATCC_BamHI
pTP10
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGCTGGATCC_BamHI
pTP11
KpnI_GGTACCATGGCGTCTTCTTCTTCTCTCACTCTCTCTCAAGCTATC
CTCTCTCGTTCTGTCCCTCGCCATGGCTCTGCCTCTTCTTCTCAACTTTC
CCCTTCTTCTCTCACTTTTTCCGGCCTTAAATCCAATCCCAATATCACCA
CCTCCCGCCGCCGTACTCCTTCCTCCGCCGCCGCCGCCGCCGTCGTAAGG
TCACCGGCGATTCGTGCCTCAGCTGCAACCGAAACCATAGAGAAAACTGA
GACTGCGGGGATCC_BamHI.

The biosynthesis site of tocopherols is, inter alia, the leaf tissue, so that leaf-specific expression of the TCMRP genes constitutes a preferred embodiment. However, this does not constitute a limitation since tocopherol biosynthesis need not be restricted to leaf tissue but can also take place in a tissue-specific manner in all other parts of the plant, in particular in fatty seeds.

Accordingly, a further preferred embodiment relates to a seed-specific expression of the TCMRP genes.

In addition, constitutive expression of the exogenous TCMRP genes is advantageous. On the other hand, inducible expression may also appear desirable.

Expression efficacy of the recombinantly expressed genes can be determined for example in vitro by shoot meristem propagation. Also, changes in the nature and level of the expression of the genes, and their effect on tocopherol biosynthesis performance, can be tested on test plants in greenhouse experiments.

Plant gene expression can also be facilitated via a chemically inducible promoter (for rewiew see Gatz 1997, Annu. Rev. Plant Physiol. Plant Mol. Biol., 48:89-108). Chemically inducible promoters are especially suitable if gene expression is wanted to occur in a time specific manner. Examples for such promoters are a salicylic acid inducible promoter (WO 95/19443), a tetracycline inducible promoter (Gatz et al., (1992) Plant J. 2, 397-404) and an ethanol inducible promoter (WO 93/21334).

Also promoters responding to biotic or abiotic stress conditions are suitable promoters such as the pathogen inducible PRP1-gene promoter (Ward et al., Plant. Mol. Biol. 22 (1993), 361-366), the heat inducible hsp80-promoter from tomato (U.S. Pat. No. 5,187,267), cold inducible alpha-amylase promoter from potato (WO9612814) or the wound-inducible pinII-promoter (EP3 75091).

Especially those promoters are preferred which confer gene expression in storage tissues and organs such as cells of the endosperm and the developing embryo.

Suitable promoters are the napin-gene promoter from rapeseed (U.S. Pat. No. 5,608,152), the USP-promoter from Vicia faba (Baeumlein et al., Mol Gen Genet, 1991, 225 (3):459-67), the oleosin-promoter from Arabidopsis (WO9845461), the phaseolin-promoter from Phaseolus vulgaris (U.S. Pat. No. 5,504,200), the Bce4-promoter from Brassica (WO9113980) or the legumin B4 promoter (LeB4; Baeumlein et al., 1992, Plant Journal, 2 (2):233-9) as well as promoters conferring seed specific expression in monocot plants like maize, barley, wheat, rye, rice etc. Suitable promoters to note are the 1pt2 or 1pt1-gene promoter from barley (WO9515389 and WO9523230) or those desribed in WO9916890 (promoters from the barley hordein-gene, the rice glutelin gene, the rice oryzin gene, the rice prolamin gene, the wheat gliadin gene, wheat glutelin gene, the maize zein gene, the oat glutelin gene, the Sorghum kasirin-gene, the rye secalin gene).

Also especially suited are promoters that confer plastid-specific gene expression as plastids are the compartment where part of the biosynthesis of amino acids, vitamins, cofactors, nutraceuticals, nucleotide or nucleosides take place. Suitable promoters such as the viral RNA-polymerase promoter are described in WO9516783 and WO9706250 and the c1 pP-promoter from Arabidopsis described in WO9946394.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TCMRP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986 and Mol et al., 1990, FEBS Letters 268:427-430.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an TCMRP can be expressed in bacterial cells such as E.coli, C. glutamicum, insect cells, fungal cells or mammalian cells (such as Chinese hamster ovary cells (CHO) or COS cells), algae, ciliates, plant cells or fungi. Other suitable host cells are known to those skilled in the art. Preferred are plant cells.

Vector DNA can be introduced into prokaryotic or eukaryotic cells via conventional transformation or transfection techniques. As used herein, the terms “transformation” and “transfection”, conjugation and transduction are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells including plant cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989) and other laboratory manuals such as Methods in Molecular Biology, 1995, Vol. 44, Agrobacterium protocols, ed: Gartland and Davey, Humana Press, Totowa, N.J.

Suitable methods are protoplast transformation by polyethylene-glycol-induced DNA uptake, the biolistic method using the gene gun—the so-called particle bombardment method, electroporation, incubation of dry embryos in DNA-containing solution, microinjection and agrobacterium-mediated gene transfer.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate or in plants that confer resistance towards a herbicide such as glyphosate or glufosinate. Nucleic acid encoding a selectable marker can be introduced into a host cell on the same vector as that encoding an TCMRP or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an TCMRP gene into which a deletion, addition or substitution has been introduced to thereby alter, e.g., functionally disrupt, the TCMRP gene. Preferably, this TCMRP gene is a Physcomitrella patens TCMRP gene, but it can be a homologue from a related plant or even from a mammalian, yeast, or insect source. In a preferred embodiment, the vector is designed such that, upon homologous recombination, the endogenous TCMRP gene is functionally disrupted (i.e., no longer encodes a functional protein; also referred to as a knock-out vector). Alternatively, the vector can be designed such that, upon homologous recombination, the endogenous TCMRP gene is mutated or otherwise altered but still encodes functional protein (e.g., the upstream regulatory region can be altered to thereby alter the expression of the endogenous TCMRP). To create a point mutation via homologous recombination also DNA-RNA hybrids can be used known as chimeraplasty known from Cole-Strauss et al. 1999, Nucleic Acids Research 27(5):1323-1330 and Kmiec Gene therapy. 19999, American Scientist. 87(3):240-247.

Whereas in the homologous recombination vector, the altered portion of the TCMRP gene is flanked at its 5′ and 3′ ends by additional nucleic acid of the TCMRP gene to allow for homologous recombination to occur between the exogenous TCMRP gene carried by the vector and an endogenous TCMRP gene in a microorganism or plant. The additional flanking TCMRP nucleic acid is of sufficient length for successful homologous recombination with the endogenous gene. Typically, several hundreds of basepairs up to kilobases of flanking DNA (both at the 5′ and 3′ ends) are included in the vector (see e.g., Thomas, K. R., and Capecchi, M. R. (1987) Cell 51: 503 for a description of homologous recombination vectors or Strepp et al., 1998, PNAS, 95 (8):43684373 for cDNA based recombination in Physcomitrella patens). The vector is introduced into a microorganism or plant cell (e.g., via polyethyleneglycol mediated DNA) and cells in which the introduced TCMRP gene has homologously recombined with the endogenous TCMRP gene are selected, using art-known techniques.

In another embodiment, recombinant microorganisms can be produced which contain selected systems which allow for regulated expression of the introduced gene. For example, inclusion of an TCMRP gene on a vector placing it under control of the lac operon permits expression of the TCMRP gene only in the presence of IPTG. Such regulatory systems are well known in the art.

A host cell of the invention, such as a prokaryotic or eukaryotic host cell in culture, can be used to produce (i.e., express) an TCMRP. An alternate method can be applied in addition in plants by the direct transfer of DNA into developing flowers via electroporation or Agrobacterium medium gene transfer. Accordingly, the invention further provides methods for producing TCMRPs using the host cells of the invention. In one embodiment, the method comprises culturing the host cell of invention (into which a recombinant expression vector encoding an TCMRP has been introduced, or into which genome has been introduced a gene encoding a wild-type or altered TCMRP) in a suitable medium until TCMRP is produced. In another embodiment, the method further comprises isolating TCMRPs from the medium or the host cell.

### C. Isolated TCMRPs

Another aspect of the invention pertains to isolated TCMRPs, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of TCMRP in which the protein is separated from cellular components of the cells in which it is naturally or recombinantly produced. In one embodiment, the language “substantially free of cellular material” includes preparations of TCMRP having less than about 30% (by dry weight) of non-TCMRP (also referred to herein as a “contaminating protein”), more preferably less than about 20% of non-TCMRP, still more preferably less than about 10% of non-TCMRP, and most preferably less than about 5% non-TCMRP.

When the TCMRP or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of TCMRP in which the protein is separated from chemical precursors or other chemicals which are involved in the synthesis of the protein. In one embodiment, the language “substantially free of chemical precursors or other chemicals” includes preparations of TCMRP having less than about 30% (by dry weight) of chemical precursors or non-TCMRP chemicals, more preferably less than about 20% chemical precursors or non-TCMRP chemicals, still more preferably less than about 10% chemical precursors or non-TCMRP chemicals, and most preferably less than about 5% chemical precursors or non-TCMRP chemicals. In preferred embodiments, isolated proteins or biologically active portions thereof lack contaminating proteins from the same organism from which the TCMRP is derived. Typically, such proteins are produced by recombinant expression of, for example, a Physcomitrella patens TCMRP in other plants than Physcomitrella patens or microorganisms such as C. glutamicum or ciliates, algae or fungi.

An isolated TCMRP or a portion thereof of the invention can participate in the metabolism of amino acids, vitamins, cofactors, nutraceuticals, nucleotides or nucleosides in Physcomitrella patens, or has one or more of the activities set forth in Table 1. In preferred embodiments, the protein or portion thereof comprises an amino acid sequence which is sufficiently homologous to an amino acid sequence of Appendix B such that the protein or portion thereof maintains the ability to participate in the metabolism of fine chemicals like amino acids, vitamins, cofactors, nutraceuticals, nucleotides, or nucleosides in Physcomitrella patens. The portion of the protein is preferably a biologically active portion as described herein. In another preferred embodiment, an TCMRP of the invention has an amino acid sequence shown in Appendix B. In yet another preferred embodiment, the TCMRP has an amino acid sequence which is encoded by a nucleotide sequence which hybridizes, e.g., hybridizes under stringent conditions, to a nucleotide sequence of Appendix A. In still another preferred embodiment, the TCMRP has an amino acid sequence which is encoded by a nucleotide sequence that is at least about 50-60%, preferably at least about 60-70%, more preferably at least about 70-80%, 80-90%, 90-95%, and even more preferably at least about 96%, 97%, 98%, 99% or more homologous to one of the amino acid sequences of Appendix B. The preferred TCMRPS of the present invention also preferably possess at least one of the TCMRP activities described herein. For example, a preferred TCMRP of the present invention includes an amino acid sequence encoded by a nucleotide sequence which hybridizes, e.g., hybridizes under stringent conditions, to a nucleotide sequence of Appendix A, and which can participate in the metabolism of tocopherols or carotenoids in Physcomitrella patens, or which has one or more of the activities set forth in Table 1.

In other embodiments, the TCMRP is substantially homologous to an amino acid sequence of Appendix B and retains the functional activity of the protein of one of the sequences of Appendix B yet differs in amino acid sequence due to natural variation or mutagenesis, as described in detail in subsection I above. Accordingly, in another embodiment, the TCMRP is a protein which comprises an amino acid sequence which is at least about 50-60%, preferably at least about 60-70%, and more preferably at least about 70-80, 80-90, 90-95%, and most preferably at least about 96%, 97%, 98%, 99% or more homologous to an entire amino acid sequence of Appendix B and which has at least one of the TCMRP activities described herein. In another embodiment, the invention pertains to a full Physcomitrella patens protein which is substantially homologous to an entire amino acid sequence of Appendix B.

Biologically active portions of an TCMRP include peptides comprising amino acid sequences derived from the amino acid sequence of an TCMRP, e.g., the an amino acid sequence shown in Appendix B or the amino acid sequence of a protein homologous to an TCMRP, which include fewer amino acids than a full length TCMRP or the full length protein which is homologous to an TCMRP, and exhibit at least one activity of an TCMRP. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an TCMRP. Moreover, other biologically active portions, in which other regions of the protein are deleted, can be prepared by recombinant techniques and evaluated for one or more of the activities described herein. Preferably, the biologically active portions of an TCMRP include one or more selected domains/motifs or portions thereof having biological activity.

TCMRPs are preferably produced by recombinant DNA techniques. For example, a nucleic acid molecule encoding the protein is cloned into an expression vector (as described above), the expression vector is introduced into a host cell (as described above) and the TCMRP is expressed in the host cell. The TCMRP can then be isolated from the cells by an appropriate purification scheme using standard protein purification techniques. Alternative to recombinant expression, an TCMRP, polypeptide, or peptide can be synthesized chemically using standard peptide synthesis techniques. Moreover, native TCMRP can be isolated from cells (e.g., endothelial cells), for example using an anti-TCMRP antibody, which can be produced by standard techniques utilizing an TCMRP or fragment thereof of this invention.

The invention also provides TCMRP chimeric or fusion proteins. As used herein, an TCMRP “chimeric protein” or “fusion protein” comprises an TCMRP polypeptide operatively linked to a non-TCMRP polypeptide. An “TCMRP polypeptide” refers to a polypeptide having an amino acid sequence corresponding to an TCMRP, whereas a “non-TCMRP polypeptide” refers to a polypeptide having an amino acid sequence corresponding to a protein which is not substantially homologous to the TCMRP, e.g., a protein which is different from the TCMRP and which is derived from the same or a different organism. Within the fusion protein, the term “operatively linked” is intended to indicate that the TCMRP polypeptide and the non-TCMRP polypeptide are fused to each other so that both sequences fulfil the proposed function addicted to the sequence used. The non-TCMRP polypeptide can be fused to the N-terminus or C-terminus of the TCMRP polypeptide. For example, in one embodiment the fusion protein is a GST-TCMRP fusion protein in which the TCMRP sequences are fused to the C-terminus of the GST sequences. Such fusion proteins can facilitate the purification of recombinant TCMRPs. In another embodiment, the fusion protein is an TCMRP containing a heterologous signal sequence at its N-terminus. In certain host cells (e.g., mammalian host cells), expression and/or secretion of an TCMRP can be increased through use of a heterologous signal sequence.

Preferably, an TCMRP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An TCMRP—encoding nucleic acid can be cloned into such an expression vector such that the fusion moiety is linked in-frame to the TCMRP.

Homologues of the TCMRP can be generated by mutagenesis, e.g., discrete point mutation or truncation of the TCMRP. As used herein, the term “homologue” refers to a variant form of the TCMRP which acts as an agonist or antagonist of the activity of the TCMRP. An agonist of the TCMRP can retain substantially the same, or a subset, of the biological activities of the TCMRP. An antagonist of the TCMRP can inhibit one or more of the activities of the naturally occurring form of the TCMRP, by, for example, competitively binding to a downstream or upstream member of the cell membrane component metabolic cascade which includes the TCMRP, or by binding to an TCMRP which mediates transport of compounds across such membranes, thereby preventing translocation from taking place.

In an alternative embodiment, homologues of the TCMRP can be identified by screening combinatorial libraries of mutants, e.g., truncation mutants, of the TCMRP for TCMRP agonist or antagonist activity. In one embodiment, a variegated library of TCMRP variants is generated by combinatorial mutagenesis at the nucleic acid level and is encoded by a variegated gene library. A variegated library of TCMRP variants can be produced by, for example, enzymatically ligating a mixture of synthetic oligonucleotides into gene sequences such that a degenerate set of potential TCMRP sequences is expressible as individual polypeptides, or alternatively, as a set of larger fusion proteins (e.g., for phage display) containing the set of TCMRP sequences therein. There are a variety of methods which can be used to produce libraries of potential TCMRP homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TCMRP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the TCMRP coding can be used to generate a variegated population of TCMRP fragments for screening and subsequent selection of homologues of an TCMRP. In one embodiment, a library of coding sequence fragments can be generated by treating a double stranded PCR fragment of an TCMRP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TCMRP.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TCMRP homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TCMRP homologues (Arkin and Yourvan (1992) PNAS 89:7811-7815; Delgrave et al (1993) Protein Engineering 6(3):327-331).

In another embodiment, cell based assays can be exploited to analyze a variegated TCMRP library, using methods well known in the art.

### D. Uses and Methods of the Invention

The nucleic acid molecules, proteins, protein homologues, fusion proteins, priners, vectors, and host cells described herein can be used in one or more of the following methods: identification of Physcomitrella patens and related organisms; mapping of genomes of organisms related to Physcomitrella patens; identification and localization of Physcomitrella patens sequences of interest; evolutionary studies; determination of TCMRP regions required for function; modulation of an TCMRP activity; modulation of the cellular production of one or more fine chemicals such as tocopherols or carotenoids. The TCMRP nucleic acid molecules of the invention have a variety of uses. First, they may be used to identify an organism as being Physcomitrella patens or a close relative thereof. Also, they may be used to identify the presence of Physcomitrella patens or a relative thereof in a mixed population of microorganisms. The invention provides the nucleic acid sequences of a number of Physcomitrella patens genes; by probing the extracted genomic DNA of a culture of a unique or mixed population of microorganisms under stringent conditions with a probe spanning a region of a Physcomitrella patens gene which is unique to this organism, one can ascertain whether this organism is present.

Further, the nucleic acid and protein molecules of the invention may serve as markers for specific regions of the genome. This has utility not only in the mapping of the genome, but also for functional studies of Physcomitrella patens proteins. For example, to identify the region of the genome to which a particular Physcomitrella patens DNA-binding protein binds, the Physcomitrella patens genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of Physcomitrella patens, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related mosses, such as Physcomitrella patens.

The TCMRP nucleic acid molecules of the invention are also useful for evolutionary and protein structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the TCMRP nucleic acid molecules of the invention may result in the production of TCMRPs having functional differences from the wild-type TCMRPs. These proteins may be improved in efficiency or activity, may be present in greater numbers in the cell than is usual, or may be decreased in efficiency or activity.

There are a number of mechanisms by which the alteration of an TCMRP of the invention may directly affect the yield, production, and/or efficiency of production of a fine chemical like tocopherols and carotenoids incorporating such an altered protein into microorganisms, algae or plants. Recovery of fine chemical compounds from large-scale cultures of C. glutamicum, ciliates, algae or fungi is significantly improved if the cell secretes the desired compounds, since such compounds may be readily purified from the culture medium (as opposed to extracted from the mass of cultured cells). In the case of plants expressing TCMRPs increased transport can lead to improved partitioning within the plant tissue and organs. By either increasing the number or the activity of transporter molecules which export fine chemicals from the cell, it may be possible to increase the amount of the produced fine chemical which is present in the extracellular medium, thus permitting greater ease of harvesting and purification or in case of plants mor efficient partitioning. Conversely, in order to efficiently overproduce one or more fine chemicals, increased amounts of the cofactors, precursor molecules, and intermediate compounds for the appropriate biosynthetic pathways are required. Therefore, by increasing the number and/or activity of transporter proteins involved in the import of nutrients, such as carbon sources (i.e., sugars), nitrogen sources (i.e., amino acids, ammonium salts), phosphate, and sulfur, it may be possible to improve the production of a fine chemical, due to the removal of any nutrient supply limitations on the biosynthetic process.

The engineering of one or more TCMRP genes of the invention may also result in TCMRPs having altered activities which indirectly impact the production of one or more desired fine chemicals from algae, plants, ciliates or fungi or other microorganims like C. glutamicum. For example, the normal biochemical processes of metabolism result in the production of a variety of waste products (e.g., hydrogen peroxide and other reactive oxygen species) which may actively interfere with these same metabolic processes (for example, peroxynitrite is known to nitrate tyrosine side chains, thereby inactivating some enzymes having tyrosine in the active site (Groves, J. T. (1999) Curr. Opin. Chem. Biol, 3(2): 226-235). While these waste products are typically excreted, cells utilized for large-scale fermentative production are optimized for the overproduction of one or more fine chemicals, and thus may produce more waste products than is typical for a wild-type cell. By optimizing the activity of one or more TCMRPs of the invention which are involved in the export of waste molecules, it may be possible to improve the viability of the cell and to maintain efficient metabolic activity. Also, the presence of high intracellular levels of the desired fine chemical may actually be toxic to the cell, so by increasing the ability of the cell to secrete these compounds, one may improve the viability of the cell.

Further, the TCMRPs of the invention may be manipulated such that the relative amounts of various lipophilic fine chemicals like for example vitamin E or carotenoids are altered. This may have a profound effect on the lipid composition of the membrane of the cell. Since each type of lipid has different physical properties, an alteration in the lipid composition of a membrane may significantly alter membrane fluidity. Changes in membrane fluidity can impact the transport of molecules across the membrane, which, as previously explicated, may modify the export of waste products or the produced fine chemical or the import of necessary nutrients. Such membrane fluidity changes may also profoundly affect the integrity of the cell; cells with relatively weaker membranes are more vulnerable abiotic and biotic stress conditions which may damage or kill the cell. By manipulating TCMRPs involved in the production of fatty acids and lipids for membrane construction such that the resulting membrane has a membrane composition more amenable to the environmental conditions extant in the cultures utilized to produce fine chemicals, a greater proportion of the cells should survive and multiply. Greater numbers of producing cells should translate into greater yields, production, or efficiency of production of the fine chemical from the culture.

The aforementioned mutagenesis strategies for TCMRPs to result in increased yields of a fine chemical are not meant to be limiting; variations on these strategies will be readily apparent to one skilled in the art. Using such strategies, and incorporating the mechanisms disclosed herein, the nucleic acid and protein molecules of the invention may be utilized to generate algae, ciliates, plants, fungi or other microorganims like C. glutamicum expressing mutated TCMRP nucleic acid and protein molecules such that the yield, production, and/or efficiency of production of a desired compound is improved. This desired compound may be any natural product of algae, ciliates, plants, fungi or C. glutamicum, which includes the final products of biosynthesis pathways and intermediates of naturally-occurring metabolic pathways, as well as molecules which do not naturally occur in the metabolism of said cells, but which are produced by a said cells of the invention.

This invention is further illustrated by the following examples which should not be construed as limiting. The contents of all references, patent applications, patents, and published patent applications cited throughout this application are hereby incorporated by reference.

### EXAMPLIFICATION

### Example 1

### General Processes

a) General Cloning Processes:

Cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA were carried out as described in Sambrook et al. (1989) (Cold Spring Harbor Laboratory Press: ISBN 0-87969-309-6) or Kaiser, Michaelis and Mitchell (1994) “Methods in Yeasr Genetics” (Cold Spring Harbor Laboratory Press: ISBN 0-87969451-3). Transformation and cultivation 21of algae such as Chlorella or Phaeodactylum are transformed as described by El-Sheekh (1999), Biologia Plantarum 42: 209-216; Apt et al. (1996), Molecular and General Genetics 252 (5): 872-9.

b) Chemicals:

The chemicals used were obtained, if not mentioned otherwise in the text, in p.a. quality from the companies Fluka (Neu-Ulm), Merck (Darmstadt), Roth (Karlsruhe), Serva (Heidelberg) and Sigma (Deisenhofen). Solutions were prepared using purified, pyrogen-free water, designated as H₂O in the following text, from a Milli-Q water system water purification plant (Millipore, Eschbom). Restriction endonucleases, DNA-modifying enzymes and molecular biology kits were obtained from the companies AGS (Heidelberg), Amersham (Braunschweig), Biometra (Göttingen), Boehringer (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Amsterdam, Netherlands). They were used, if not mentioned otherwise, according to the manufacturer's instructions.

### c) Plant Material

For this study, plants of the species Physcomitrella patens (Hedw.) B.S.G. from the collection of the genetic studies section of the University of Hamburg were used. They originate from the strain 16/14 collected by H. L. K. Whitehouse in Gransden Wood, Huntingdonshire (England), which was subcultured from a spore by Engel (1968, Am J Bot 55, 438446). Proliferation of the plants was carried out by means of spores and by means of regeneration of the gametophytes. The protonema developed from the haploid spore as a chloroplast-rich chloronema and chloroplast-low caulonema, on which buds formed after approximately 12 days. These grew to give gametophores bearing antheridia and archegonia. After fertilization, the diploid sporophyte with a short seta and the spore capsule resulted, in which the meiospores mature.

### d) Plant Growth

Culturing was carried out in a climatic chamber at an air temperature of 25□C and light intensity of 55 micromols-1m-2 (white light; Philips TL 65W/25 fluorescent tube) and a light/dark change of 16/8 hours. The moss was either modified in liquid culture using Knop medium according to Reski and Abel (1985, Planta 165, 354-358) or cultured on Knop solid medium using 1% oxoid agar (Unipath, Basingstoke, England).

The protonemas used for RNA and DNA isolation were cultured in aerated liquid cultures. The protonemas were comminuted every 9 days and transferred to fresh culture medium.

### Example 2

### Total DNA Isolation From Plants

The details for the isolation of total DNA relate to the working up of one gram fresh weight of plant material.

CTAB buffer: 2% (w/v) N-cethyl-N,N,N-trimethylammonium bromide (CTAB); 100 mM Tris HCl pH 8.0; 1.4 M NaCl; 20 mM EDTA.

N-Laurylsarcosine buffer: 10% (w/v) N-laurylsarcosine; 100 mM Tris HCl pH 8.0; 20 mM EDTA.

The plant material was triturated under liquid nitrogen in a mortar to give a fine powder and transferred to 2 ml Eppendorf vessels. The frozen plant material was then covered with a layer of 1 ml of decomposition buffer (1 ml CTAB buffer, 100 ml of N-laurylsarcosine buffer, 20 ml of b-mercaptoethanol and 10 ml of proteinase K solution, 10 mg/ml) and incubated at 60 C for one hour with continuous shaking. The homogenate obtained was distributed into two Eppendorf vessels (2 ml) and extracted twice by shaking with the same volume of chloroform/isoamyl alcohol (24:1). For phase separation, centrifugation was carried out at 8000×g and RT for 15 min in each case. The DNA was then precipitated at −70 C. for 30 min using ice-cold isopropanol. The precipitated DNA was sedimented at 4 C and 10,000 g for 30 min and resuspended in 180 ml of TE buffer (Sambrook et al., 1989, Cold Spring Harbor Laboratory Press: ISBN 0-87969-309-6). For further purification, the DNA was treated with NaCl (1.2 M final concentration) and precipitated again at −70 C. for 30 min using twice the volume of absolute ethanol. After a washing step with 70% ethanol, the DNA was dried and subsequently taken up in 50 ml of H₂O+RNAse (50 mg/ml final concentration). The DNA was dissolved overnight at 4 C and the RNAse digestion was subsequently carried out at 37 C for 1 h. Storage of the DNA took place at 4 C.

### Example 3

### Isolation of Total RNA and Poly-(A)+RNA From Plants

For the investigation of transcripts, both total RNA and poly-(A)⁺RNA were isolated. The total RNA was obtained from wild-type 9d old protonemata following the GTC-method (Reski et al. 1994, Mol. Gen. Genet., 244:352-359).

Isolation of PolyA+RNA was isolated using Dyna Beads® (Dynal, Oslo) Following the instructions of the manufacturers protocol.

After determination of the concentration of the RNA or of the poly-(A)+RNA, the RNA was precipitated by addition of 1/10 volumes of 3 M sodium acetate pH 4.6 and 2 volumes of ehanol and stored at −70 C.

### Example 4

### cDNA Library Construction

For cDNA library construction first strand synthesis was achieved using Murine Leukemia Virus reverse transcriptase (Roche, Mannheim, Germany) and olido-d(T)-primers, second strand synthesis by incubation with DNA polymerase I, Klenow enzyme and RNAseH digestion at 12° C. (2 h), 16° C. (1 h) and 22° C. (1h). The reaction was stopped by incubation at 65° C. (10 min) and subsequently transferred to ice. Double stranded DNA molecules were blunted by T4-DNA-polymerase (Roche, Mannheim) at 37° C. (30 min). Nucleotides were removed by phenol/chloroform extraction and Sephadex-G50 spin columns. EcoRI adapters (Pharmacia, Freiburg, Germany) were ligated to the cDNA ends by T4-DNA-ligase (Roche, 12° C., overnight) and phosphorylated by incubation with polynucleotide kinase (Roche, 37° C., 30 min). This mixture was subjected to separation on a low melting agarose gel. DNA molecules larger than 300 basepairs were eluted from the gel, phenol extracted, concentrated on Elutip-D-columns (Schleicher and Schuell, Dassel, Germany) and were ligated to vector arms and packed into lambda ZAPII-phages or lambda ZAP-Express phages using the Gigapack Gold Kit (Stratagene, Amsterdam, Netherlands) using material and following the instructions of the manufacturer.

### Example 5

### Identification of Genes of Interest

Gene sequences can be used to identify homologous or heterologous genes from cDNA or genomic libraries.

Homologous genes (e. g. full length CDNA clones) can be isolated via nucleic acid hybridization using for example cDNA libraries: Depended on the abundance of the gene of interest 100 000 up to 1 000 000 recombinant bacteriophages are plated and transferred to a nylon membrane. After denaturation with alkali, DNA is immobilized on the membrane by e. g. UV cross linking. Hybridization is carried out at high stringency conditions. In aqueous solution hybridization and washing is performed at an ionic strength of 1 M NaCl and a temperature of 68□C. Hybridization probes are generated by e. g. radioactive (³²P) nick transcription labeling (Amersham Ready Prime). Signals are detected by exposure to x-ray films.

Partially homologous or heterologous genes that are related but not identical can be identified analog to the above described procedure using low stringency hybridization and washing conditions. For aqueous hybridization the ionic strength is normally kept at 1 M NaCl while the temperature is progressively lowered from 68 to 42□C.

Isolation of gene sequences with homologies only in a distinct domain of (for example 20 aminoacids) can be carried out by using synthetic radio labeled oligonucleotide probes. Radio labeled oligonucleotides are prepared by phosphorylalation of the 5′-prime end of two complementary oligonucleotides with T4 polynucleotede kinase. The complementary oligonucleotides are annealed and ligated to form concatemers. The double stranded concatemers are than radiolabled by for example nick transcription. Hybridization is normally performed at low stringency conditions using high oligonucleotide concentrations.

Oligonucleotide hybridization solution:

6×SSC

0.01 M sodium phosphate

1 mM EDTA (pH 8)

0.5% SDS

100 μg/ml denaturated salmon sperm DNA

0.1% nonfat dried milk

During hybridization temperature is lowered stepwise to 5-10□C below the estimated oligonucleotid Tm.

Further details are described by Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons.

### Example 6

### Identification of Genes of Interest by Screening Expression Libraries With Antibodies

C-DNA sequences can be used to produce recombinant protein for example in E. coli (e.g. Qiagen QIAexpress pQE system). Recombinant proteins are than normally affinity purified via Ni-NTA affinity chromatoraphy (Qiagen). Recombinant proteins are than used to produce specific antibodies for example by using standard techniques for rabbit immunization. Antibodies are affinitypurified using a Ni-NTA column saturated with the recombinant antigen as described by Gu et al., (1994)BioTechniques 17: 257-262. The antibody can than be used to screen expression cDNA libraries to identify homologous or heterologous genes via an immunological screening (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons).

### Example 7

### Northern-Hybridization

For RNA hybridization, 20 mg of total RNA or 1 mg of poly-(A)+RNA were separated by gel electrophoresis in 1.25% strength agarose gels using formaldehyde as described in Amasino (1986, Anal. Biochem. 152, 304), transferred by capillary attraction using 10×SSC to positively charged nylon membranes (Hybond N+, Amersham, Braunschweig), immobilized by UV light and prehybridized for 3 hours at 68° C. using hybridization buffer (10% dextran sulfate w/v, 1 M NaCl, 1% SDS, 100 mg of herring sperm DNA). The labeling of the DNA probe with the “Highprime DNA labeling kit” (Roche, Mannheim, Germany) was carried out during the prehybridization using alpha-³²p dCTP (Amersham, Braunschweig, Germany). Hybridization was carried out after addition of the labeled DNA probe in the same buffer at 68° C. overnight. The washing steps were carried out twice for 15 min using 2×SSC and twice for 30 min using 1×SSC, 1% SDS at 68° C. The exposure of the sealed-in filters was carried out at −70° C. for a period of 1-14d.

### Example 8

### DNA Sequencing

CDNA libraries as described in Example 4 were used for DNA sequencing according to standard methods, in particular by the chain termination method using the ABI PRISM Big Dye Terminator Cycle Sequencing Ready Reaction Kit (Perkin-Elmer, Weiterstadt, Germany). Random Sequencing was carried out subsequent to preparative plasmid recovery from cDNA libraries via in vivo mass excision and retransformation of DH10B on agar plates (material and protocol details from Stratagene, Amsterdam, Netherlands. Plasmid DNA was prepared from overnight grown E. coli cultures grown in Luria-Broth medium containing ampicillin (see Sambrook et al. (1989) (Cold Spring Harbor Laboratory Press: ISBN 0-87969-309-6)) on a Qiagene DNA preparation robot (Qiagen, Hilden) according to the manufacturers protocols. Sequencing primers with the following nucleotide sequences were used:

5′-CAGGAAACAGCTATGACC-3′
5′-CTAAAGGGAACAAAAGCTG-3′
5′-TGTAAAACGACGGCCAGT-3′

### Example 9

### Plasmids for Plant Transformation

For plant transformation binary vectors such as pBinAR-TkTp-9 (Badur, 1998 PhD thesis, Georg August University of Göttingen, Germany, “Molecular and functional analysis of isoenzymes for example of fructose-1,6-bisphosphate aldolase, phosphoglucose-isomerase and 3-deoxy-D-arabino-heptusolonate-7-phosphate synthase” [“Molekularbiologische und funktionelle Analyse von pflanzlichen Isoenzymen am Beispiel der Fructose-1,6-bisphosphat Aldolase, Phosphoglucose-Isomerase und der 3-Deoxy-D-Arabino-Heptusolonat-7-Phosphat Synthase”]) can be used. This vector is a derivative of pBinAR (Höfgen and Willmitzer, Plant Science 66(1990), 221-230) and contains the CaMV (cauliflower mosaic virus) 35S promoter (Franck et al., 1980), the termination signal of the octopine synthase gene (Gielen et al., 1984) and the DNA sequence encoding the transit peptide of the Nicotiana tabacum plastid transketolase. Construction of the binary vectors can be performed by ligation of the cDNA in sense or antisense orientation into the T-DNA.

5′-prime to the cDNA a plant promotor activates transcription of the cDNA. A polyadenylation sequence is located 3′-prime to the cDNA.

Tissue specific expression can be archived by using a tissue specific promotor. For example seed specific expression can be archived by cloning the napin or USP promotor 5-prime to the cDNA. Also any other seed specific promotor element can be used. For constitutive expression within the whole plant the CaMV 35S promotor can be used.

The expressed protein can be targeted to a cellular compartment using a signal peptide, for example for plasids, mitochondria or endoplasmatic reticulum (Kermode, Crit. Rev. Plant Sci. 15, 4 (1996), 285423). The signal peptide is cloned 5 ′-prime in frame to the cDNA to archive subeellular localization of the fusionprotein.

Nucleic acid molecules from Physcomitrella are used for a direct gene knock-out by homologous recombination. Therefore Physcometrella sequences are usefull for functional genomic approaches. The technique is described by Strepp et al., Proc. Natl. Acad. Sci. USA,1998, 95: 4369 -4373; Girke et al. (1998), Plant Journal 15: 39-48; Hofmann et al. (1999) Molecular and General Genetics 261: 92-99.

### Example 10

### Transformation of Agrobacterium

Agrobacterium mediated plant transformation can be performed using for example the GV3101(pTCMRP90) (Koncz and Schell, Mol. Gen.Genet. 204 (1986), 383-396) or LBA4404 (Clontech) Agrobacterium tumefaciens strain. Transformation can be performed by standard transformation techniques (Deblaere et al., Nucl. Acids. Tes. 13 (1984),4777-4788).

### Example 11

### Plant Transformation

Agrobacterium mediated plant transformation has been performed using standard transformation and regeneration techniques (Gelvin, Stanton B.; Schilperoort, Robert A, “Plant Molecular Biology Manual”, 2nd Ed. —Dordrecht: Kluwer Academic Publ., 1995. —in Sect., Ringbuc Zentrale Signatur: BT11-P ISBN 0-7923-2731-4; Glick, Bernard R.; Thompson, John E., “Methods in Plant Molecular Biology and Biotechnology”, Boca Raton: CRC Press, 1993. -360 S., ISBN 0-8493-5164-2).

For example rapeseed can be transformed via cotyledon or hypocotyl transformation (Moloney et al., Plant cell Report 8 (1989), 238-242; De Block et al., Plant Physiol. 91 (1989, 694-701). Use of antibiotica for agrobacterium and plant selection depends on the binary vector and the agrobacterium strain used for transformation. Rapeseed selection is normally performed using kanamycin as selectable plant marker.

Agrobacterium mediated gene transfer to flax can be performed using for example a technique described by Mlynarova et al. (1994), Plant Cell Report 13: 282-285.

Transformation of soybean can be performed using for example a technique described in EP 0424 047, U.S. Pat. No. 322,783 (Pioneer Hi-Bred International) or in EP 0397 687, U.S. Pat. No. 5,376,543, U.S. Pat. No. 5,169,770 (University Toledo).

Plant transformation using particle bombardment, Polyethylene Glycol mediated DNA uptake or via the Silicon Carbide Fiber technique is for example described by Freeling and Walbot “The maize handbook” (1993) ISBN 3-540-97826-7, Springer Verlag New York).

### Example 12

### In vivo Mutagenesis

In vivo mutagenesis of microorganisms can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 277-2294, ASM: Washington.) Such strains are well known to those skilled in the art. he use of such strains is illustrated, for example, in Greener, A. and Callahan, M. (1994) Strategies 7: 32-34. Transfer of mutated DNA molecules into plants is preferably done after selection and testing in microorganisms. Transgenic plants are generated according to various examples within the exemplification of this document.

### Example 13

### DNA Transfer Between Escherichia coli and Corynebacterium glutamicum

Several Corynebacterium and Brevibacterium species contain endogenous plasmids (as e.g., pHM1519 or pBL1) which replicate autonomously (for review see, e.g., Martin, J. F. et al. (1987) Biotechnology, 5:137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide variety of shuttle vectors which replicate in both E. coli and C. glutamicum, and which can be used for several purposes, including gene over-expression (for reference, see e.g., Yoshihama, M. et al. (1985) J. Bacteriol. 162:591-597, Martin J. F. et al. (1987) Biotechnology, 5:137-146 and Eikmanns, B. J. et al. (1991) Gene, 102:93-98). Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebl, E. et al. (1989) FEMS Microbiol. Letters, 53:399-303) and in cases where special vectors are used, also by conjugation (as described e.g. in Schäfer, A et al. (1990) J. Bacteriol. 172:1663-1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. colistrain, such as NM522 (Gough & Murray (1983) J Mol. Biol. 166:1-19).

### Example 14

### Assessment of the Expression of a Recombinant Gene Product in a Transformed Organism

The activity of a recombinant gene product in the transformed host organism has been measured on the transcriptional or/and on the translational level.

A useful method to ascertain the level of transcription of the gene (an indicator of the amount of mRNA available for translation to the gene product) is to perform a Northern blot (for reference see, for example, Ausubel et al. (1988) Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the transformed gene. Total cellular RNA can be prepared from cells, tissues or organs by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or calorimetric label which may be readily detected. The presence and quantity of label observed indicates the presence and quantity of the desired mutant protein present in the cell.

### Example 15

### Growth of Genetically Modified Corynebacterium Glutamicum—Media and Culture Conditions

Genetically modified Corynebacteria are cultured in synthetic or natural growth media. A number of different growth media for Corynebacteria are both well-known and readily available (Lieb et al. (1989) App. Microbiol Biotechnol., 32:205-210; von der Osten et al. (1998) Biotechnology Letters, 11:11-16; Patent DE 4,120,867; Liebl (1992) “The Genus Corynebacterium, in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate- salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (brain heart infusion, DIFC) or others.

All medium components are sterilized, either by heat (20 minutes at 1.5 bar and 121□C) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15□C and 45□C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in nmicrotiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100 -300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of 0.5-1.5 using cells grown on agar plates, such as CM plates (10 g/l glucose, 2,5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 3□C. Inoculation of the media is accomplished by either introduction of a saline suspension of C. glutamicum cells from CM plates or addition of a liquid preculture of this bacterium.

### Example 16

### In vitro Analysis of the Function of Physcomitrella Genes in Transgenic Organisms

The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: New York; Walsh, (1979) Enzymatic Reaction Mechanisms. Freeman: San Francisco; Price, N. C., Stevens, L. (1982) Fundamentals of Enzymology. Oxford Univ. Press: Oxford; Boyer, P.D., ed. (1983) The Enzymes, 3^{rd} ed. Academic Press: New York; Bisswanger, H., (1994) Enzymkinetik, 2^{nd} nd ed. VCH: Weinheim (ISBN 3527300325); Bergmeyer, H. U., Bergmeyer, J., Graβ1, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### Example 17

### Analysis of Impact of Recombinant Proteins on the Production of the Desired Product

The effect of the genetic modification in plants, algae, C. glutamicum, fungi, cilates or on production of a desired compound (such as vitamins) can be assessed by growing the modified microorganism or plant under suitable conditions (such as those described above) and analyzing the medium and/or the cellular component for increased production of the desired product (i.e. fine chemicals). Such analysis techniques are well known to one skilled in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. et al. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product in plant cells, microorganisms and algae, it is also possible to analyze other components of the metabolic pathways utilized for the production of the desired compound, such as intermediates and side-products, to determine the overall efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

Material to be analyzed can be disintegrated via sonification, glass milling, liquid nitrogen and grinding or via other applicable methods. The material has to be centrifuged after disintegration.

Vitamin E:

The determination of tocopherols in cells has been either conducted according to Kurilich et al 1999, J. Agric. Food. Chem. 47: 1576-1581 or alternatively as described in Tani Y and Tsumura H 1989 (Agric. Bio. Chem. 53: 305-312).

Carotenoids:

The large scale production and purification of carotenoids implies a solution for separation of lipophilic impurities from the host cell which have to be separated from the carotenoids. On a production scale the material has to be desintegrated for the production of oleoresins via centrifugation as known skilled in the art from various production processes or via desintegration followed by evaporation and extraction. Acetone or hexane extraction for 8-12 hours in the dark to avoid carotenoid break down. After removal of the solvent the residue is dissolved in a diethylether-hexane mixture or, in case of hydroxycarotenoids, in acetone-petrol and purified via silica-gel column. Suitable solvent mixtures are diethylether:hexane or petrol (1:4 v/v) for carotenes and acetone:hexane or petrol (1:4 v/v) for hydroxycarotenoids. To determine carotenoid purity in isolated fractions HPLC techniques are most appropriate (Linden et al., FEMS Microbiol. Let. 106:99-104; Piccaglia et al., 1998; Industrial Crops and Products 8:45-51 and references therein).

### Example 18

### Purification of the Desired Product From Transformed Organisms

Recovery of the desired product from plants material or fungi, algae, cilates or C. glutamicum cells or supematant of the above-described cultures can be performed by various methods well known in the art. If the desired product is not secreted from the cells. The cells, can be harvested from the culture by low-speed centrifugation, the cells can be lysed by standard techniques, such as mechanical force or sonification. Organs of plants can be separated mechanically from other tissue or organs. Following homogenization cellular debris is removed by centrifugation, and the supernatant fraction containing the soluble proteins is retained for further purification of the desired compound. If the product is secreted from desired cells, then the cells are removed from the culture by low-speed centrifugation, and the supemate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One skilled in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: New York (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60: 133-140; Malakhova et al. (1996) Biotekhnologiya 11: 27-32; and Schmidt et al. (1998) Bioprocess Engineer. 19: 67-70. Ulmann's Encyclopedia of Industrial Chemistry, (1996) vol. A27, VCH: Weinheim, p. 89-90, p. 521-540, p. 540-547, p. 559-566, 575-581 and p. 581-587; Michal, G. (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

### Example 19

### Generation of Transgenic Brassica napus Plants

The generation of transgenic oilseed rape plants followed in principle a procedure of Bade, J. B. and Damm, B. (in Gene Transfer to Plants, Potrykus, I. and Spangenberg, G., eds, Springer Lab Manual, Springer Verlag, 1995, 30-38), which also indicates the composition of the media and buffers used. transformations were done with the Agrobacterium tumefaciens strains EHA105 and GV3101, respectively. Recombinate plasmids were used for transformation. Seeds of Brassica napus var. Westar were surface-sterilized with 70% ethanol (v/v), washed for 10 minutes at 55□C in water, incubated for 20 minutes in 1% strength hypochlorite solution (25% v/v Teepol, 0.1% v/v Tween 20) and washed six times with sterile water for in each case 20 minutes. The seeds were dried for three days on filter paper and 10-15 seeds were germinated in a glass flask containing 15 ml of germination medium. Roots and apices were removed from several seedlings (approx. size 10 cm), and the hypocotyls which remained were cut into sections of approx. length 6 mm. The approx. 600 explants thus obtained were washed for 30 minutes in 50 ml of basal medium and transferred into a 300 ml flask. After addition of 100 ml of callus induction medium, the cultures were incubated for 24 hours at 100 rpm.

An overnight culture of agrobacterial strain was set up in Luria broth medium supplemented with kanamycin (20 mg/1) at 29□C, and 2 ml of this were incubated in 50 ml of Luria broth medium without kanamycin for 4 hours at 29□C until an OD₆₀₀ of 0.4-0.5 was reached. After the culture had been pelleted for 25 minutes at 2000 rpm, the cell pellet was resuspended in 25 ml of basal medium. The bacterial concentration of the solution was brought to an OD₆₀₀ of 0.3 by adding more basal medium.

The callus induction medium was removed from the oilseed rape explants using sterile pipettes, 50 ml of agrobacterial solution were added, and the reaction was mixed carefully and incubated for 20 minutes. The agrobacterial suspension was removed, the oilseed rape explants were washed for 1 minute with 50 ml of callus induction medium, and 100 ml of callus induction medium were subsequently added. Coculturing was carried out for 24 hours on an orbital shaker at 100 rpm. Coculturing was stopped by removing the callus induction medium and explants were washed twice for in each case 1 minute with 25 ml and twice for 60 minutes with in each case 100 ml of wash medium at 100 rpm. The wash medium together with the explants was transferred into 15 cm Petri dishes, and the medium was removed using sterile pipettes.

For regeneration, in each case 20-30 explants were transferred into 90 mm Petri dishes containing 25 ml of shoot induction medium supplemented with kanamycin. The Petri dishes were sealed with 2 layers of Leukopor and incubated at 25□C and 2000 lux at photoperiods of 16 hours light/8 hours darkness. Every 12 days, the calli which developed were transferred to fresh Petri dishes containing shoot induction medium. All further steps for the regeneration of intact plants were carried out as described by Bade, J. B and Damm, B. (in Gene Transfer to Plants, Potrykus, I. and Spangenberg, G., eds, Springer Lab Manual, Springer Verlag, 1995, 30-38).

### Example 20

### Generation of Transgenic Nicotiana tabacum plants

10 ml of YEB medium supplemented with antibiotic (5 g/l beef extract, 1 g/l yeast extract, 5 g/l peptone, 5 g/l sucrose and 2 mM MgSO₄) were inoculated with a colony of Agrobacterium tumefaciens and the culture was grown overnight at 28□C. The cells were pelleted for 20 minutes at 40□C, 3500 rpm, using a bench-top centrifuge and then resuspended under sterile conditions in fresh YEB medium without antibiotics. The cell suspension was used for the transformation.

The sterile-grown wild-type plants were obtained by vegetative propagation. To this end, only the tip of the plant was cut off and transferred to fresh 2MS medium in a sterile preserving jar. As regards the rest of the plant, the hairs on the upper side of the leaves and the central veins of the leaves were removed. Using a razor blade, the leaves were cut into sections of approximate size 1 cm². The agrobacterial culture was transferred into a small Petri dish (diameter 2 cm). The leaf sections were briefly drawn through this solution and placed with the underside of the leaves on 2MS medium in Petri dishes (diameter 9 cm) in such a way that they touched the medium. After two days in the dark at 25□C, the explants were transferred to plates with callus induction medium and warmed at 28□C in a controlled-environment cabinet. The medium had to be changed every 7-10 days. As soon as calli formed, the explants were transferred into sterile preserving jars onto shoot induction medium supplemented with claforan (0.6% BiTec-Agar (g/v), 2.0 mg/l zeatin ribose, 0.02 mg/l naphthylacetic acid, 0.02 mg/l of gibberellic acid, 0.25 g/ml claforan, 1.6% glucose (g/v) and 50 mg/l kanamycin). Organogenesis started after approximately one month and it was possible to cut off the shoots which had formed. The shoots were grown on 2MS medium supplemented with claforan and selection marker. As soon as substantial root ball had developed, it was possible to pot up the plants in seed compost.

### Example 21

### Generation of Transgenic A. thaliana Plants

Wild-type A. thaliana plants (Columbia) were transformed with the Agrobacterium tumefaciens strain (EHA105) on the basis of a modified method (Steve Clough and Andrew Bent. Floral dip: a simplified method for Agrobacterium mediated transformation of A. thaliana. Plant J 16(6):735-43, 1998) of the vacuum infiltration method as described by Bechtold and coworkers (Bechtold, N. Ellis, J. and Pelltier, G., in planta Agrobacterium-mediated gene transfer by infiltration of adult A. thaliana plants. CRAcad Sci Paris, 1993. 1144(2):204-212).

### Example 22

### Characterization of the Transgenic Plants

To confirm that expression of the TCMRP genes affected vitamin E biosynthesis in the transgenic plants, the tocopherol and tocotrienol contents in leaves and seeds of the plants (Arabidopsis. thaliana, Brassica napus and Nicotiana tabacum) which had been transformed with the above-described constructs were analyzed. To this end, the transgenic plants were grown in the greenhouse, and plants which express the gene encoding the TCMRP polypeptides were identified at Northern level. The tocopherol content and the tocotrienol content in leaves and seeds of these plants were determined. In all cases, the tocopherol or tocotrienol concentration is elevated in comparison with untransformed plants.

### Example 23

### Isolation of Full Length Physcomitrella patens 78_ppprot1_{—}092_E12-260 cDNA

Utilizing the partial sequence of the Physcomitrella patens clone 78_ppprot1_{—}092_E 12 as probe, an Physcomitrella patens cDNA library was screened by nucleic acid hybridization for full length cDNAs.

A large number of hybridizing clones were isolated. The isolated cDNA 78_ppprot1 _{—}092_E12-260 (1968 bp) was sequenced completely. 78_ppprot1 _{—}092_E12-260 encodes a 492 amino acid protein.

### Example 24

### Amplification of the Coding Sequence (ORF) of the Full Length Clone 78_ppprot1_{—}092_E12-260

The coding sequence (ORF) of the 78_ppprot1_{—}092_E12-260 clone was amplified using polymerase chain reaction (PCR). The sequence of the resultant PCR fragment is designated 092-260 cds. The forward and reverse primers (78_ppprot1_{—}092_E125′ and 78_ppprot1_{—}092_E123′, respectively) were designed to add a BamHI site to the 5′ and 3′ end of the resulting amplication product.

Forward primer 78ppprot1_{—}092_E12-260z_{—}5′:

GGATCCATCATGGCGGTCAATACCGAGC

Reverse primer 78_ppprot1_{—}092_E12-260_{—}3′:

GGATCCCAAGATCATAATGCCTTGTAGGC

The PCR reaction was conducted in a 50 μl reaction mixture, containing dNTPs (0.2 mM each), 1,5 mM Mg(OAc)₂, 40 pmol 78_pprotl-092_E125′, 40 pmol 78_ppprot1_{—}092_E123′, 15 μ3,3×rTth DNA Polymerase XLPuffer (PE Applied Biosystems), 5U rTth DNA Polymerase XL (PE Applied Biosystems).

The following conditions were used:

step 1: 5 minutes 94° C. (denaturation)

step 2: 3 seconds 94° C.(denaturation)

step 3: 2 minutes 65° C. (annealing)

step 4: 1 minutes 72° C. (elongation)

40 cycles step 2-4

step: 5: 10 minutes 72° C.

The resulting PCR fragment was cloned into the PCR cloning vector pGEM-T (Promega) as described in the instructions. The recombinant plasmid (pGEM-Teasy/092-260 cds) was sequenced to confirm the correct amplification.

### Example 25

### Demonstration of 2-methyl-6-phytylplastoquinol-methyltransferase Activity (TMT type II) of 78_ppprot1_{—}092_E12 cDNA Clone by Expression and Biochemical Analysis in E.coli

In order to demonstrate that the clone 78_ppprot1_{—}092_E12-260 encodes a protein involved in tocopherol biosynthesis the cDNA 092-260 cds (cds=coding sequence amplified as described above) was expressed in E.coli and tested for 2-methyl-6-phytylplastoquinol-methyltransferase activity.

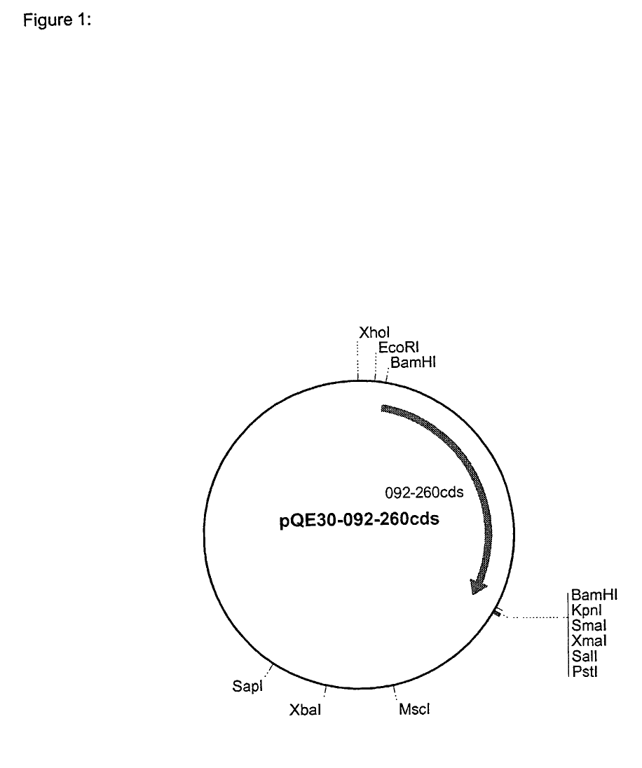

Hence, the 092-260 cds BamHI fragment was subcloned in the correct reading frame into the BamHI site of the E.coli pQE30 expression vector (QIAexpress Kit, Qiagen). The resulting plasmid (designated pQE30-092-260 cds, see FIG. 1) was used to transform the E.coli expression host strain M15[pREP4].

An E.coli colony transformed with the plasmid pQE30-092-260 cds was used to inoculate an overnight culture of Luria broth containing 200 μg/ml ampicillin. In the morning an aliquot of this culture was used to inoculate a 100 ml culture of Luria broth containing 200 μg/ml ampicillin. This culture was incubated in a shaking incubator at 28° C. until the OD₆₀₀ of the culture reached 0.4, at which time isopropyl-β-D-thiogalactopyranosid (IPTG) was added to obtain a final concentration of 0.4 mM IPTG. The culture was incubated for additional three hours at 28° C. Afterwards the cells were harvested by centrifugation at 8000g.

The pellet was resuspended in 600 μl lysis buffer (approximately 1-1.5 ml/g cell pellet, 10 mM HEPES KOH pH 7.8, 5 mM Dithiothreito1 (DTT), 0.24 M Sorbito1). Subsequently Phenylmethylsulfonat (PMSF) was added to a final concentration of 0.15 mM and the homogenate was incubated on ice for 10 minutes.

The cells were lysed by sonification with a microtip sonicator using several 10 second pulses.

After adding Triton X100 (f.c. 0.1%) the homogenate was incubated for 30 minutes on ice, and subjected to centrifugation at 25000 g for 30 minutes. The supernatant was saved for methyltransferase assays.

The 2-methyl-6-phytylplastoquinol-methyltransferase assay was performed in a 500 μl volume containing 135 μl (about 300-600 μg total protein) E.coli extract expressing the 092-260 CDNA (prepared as described above), 200 μl (125 mM) Tricine-NaOH pH 8.0, 100 μl (1.25 mM) Sorbitol, 10 μl (50 mM) MgCl₂ and 20μl (250 mM) Ascorbate, 15 μl (0.46 mM ¹⁴C-methyl-S-adenosylmethionine (SAM)) as methyl group-donor and 2-methyl-6-phytylplastoquinol as substrate. The reaction was incubated for four hours at 25° C. in the dark.

The reaction was stopped by adding 7501 μl Chloroform/Methanol (1:2)+1501 μl 0.9% NaCl. The tube were mixed thoroughly, the phases were separated by centrifugation and the upper part was discarded. The lower part was transferred to a new tube and vaporized under a stream of nitrogen.

The dried residue was resuspended in 20 μl ether and spotted onto a silica thin layer-chromatography (TLC) plate. The TLC plate was exposed to a phosphoimager screen.

The result showed that the 092-260cds protein expressed was able to methylate 2-methyl-6-phytylplastoquinol. No radioactive labelling of the substrate was observed in assays using extracts from control cells.

### Example 26

### Construction of Vectors for Expressing the Physcomitrella 2-Methyl-6-phytylplastoquinol-methyltransferase in A. thatiana and Other Plants for Altering the Content of Tocopherols

In order to manipulate the Vitamin E levels in seeds, the cDNA clone 78_ppprot1_{—}092_E12-260 encoding the Physcomitrella patens 2-methyl-6-phytylplastoquinol-methyltransferase was expressed under the control of a seed specific promoter in transgenic A.thaliana plants. The seed-specific plant gene expression plasmid was constructed using a pBin19 (Bevan, Nucleic Acid Research 12: 8711-8720, 1984) derivative. The plasrnid contains the Vicia faba seed specific promotor from the Legumin B4 gene (Bäumlein et al., Nucleic Acids Research 14: 2707-2719, 1996), the sequence encoding the transit peptide of the N. tabacum Transketolase (TkTp) (Badur, R., 1998, PhD thesis, Georg August University of Göttingen, Germany, “Molecular and functional analysis of isoenzymes for example of fructose-1,6-bisphosphate aldolase, phosphoglucose-isomerase and 3-deoxy-D-arabino-heptusolonate-7-phosphate synthase” [“Molekularbiologische und funktionelle Analyse von pflanzlichen Isoenzymen am Beispiel der Fructose-1,6-bisphosphat Aldolase, Phosphoglucose-Isomerase und der 3-Deoxy-D-Arabino-Heptusolonat-7-Phosphat Synthase”]) and the transcriptional termination sequence from the octopin synthase gene (Gielen et al., EMBO J. 3: 835-846, 1984). The cDNA 092-260 cds was cloned in sense orientation as a BamnHI fragment into the BamHI site of the pBin-LePTkTp9 vector. The created plasmid was designated pBinLePTkTp9-092-260 cds. Due to the cloning in the correct reading frame, the cDNA 092-260 cds was fused to the TkTp transit peptide, which governs the translocation of the 092-260 cds protein into plastids.

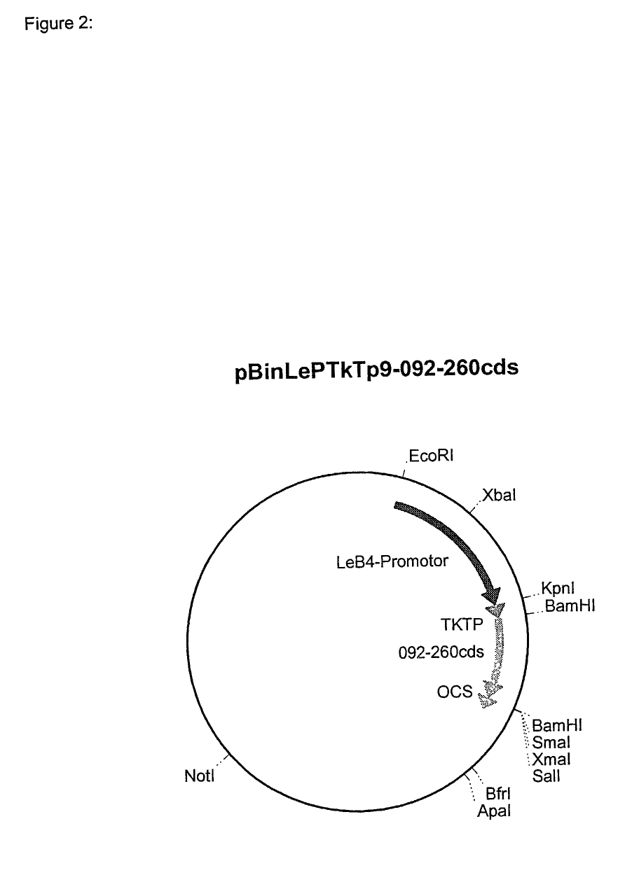

A recombinant plasmid was obtained and designated pBin-LePTkTp9-092-260 cds (see FIG. 2). This seed-specific 78_ppprot1_{—}092_E12-260 plant gene expression construct (pBin-LePTkTp9-092-260 cds) was used to transform wild type, A.thaliana plants

### Example 27

### Isolation of Full Length Physcomitrella patens 78_ppprot1_{—}087_E12-259 cDNA

Utilizing the partial sequence of the Physcomitrella patens clone 78_ppprot1_{—}087_E12 as probe, an Physcomitrella patens cDNA library was screened by nucleic acid hybridization for full length cDNAs.

A large number of hybridizing clones were isolated. The isolated cDNA 78_ppprot1_{—}087_E12-259 (1867 bp) was sequenced completely. 78_ppprot1_{—}087_E12-259 encodes a 371 amino acid protein.

### Example 28

Amplification of the coding sequence (ORF) of the full length clone 78_pprot1_{—}087_E12-259

The coding sequences (ORF) of the 78_ppprot1_{—}087_E12-259 clone with homology to the γ-Tocopherol-methyltransferases (designated 087-259Cterm) was amplified using polymerase chain reaction (PCR). The forward and reverse primers (78_ppprot1_{—}087_E12-259_{—}5′ and 78_ppprot1_{—}087_E12-259_{—}3′, respectively) were designed to add a BamHI site to the 5′ and 3′ end of the resulting amplication product.

Forward primer 78_pprotl_{—}087_E12-259_{—}5′

GGATCCCGGACGGAGCCGGAGCTTTACG

Reverse primer 78_ppprot1_{—}087_E12-259_{—}3′

GGATCCCTACTAGCGGAGACCTCAATCC

The PCR reaction was conducted in a 50 82 l reaction mixture, containing dNTPs (0.2 mM each), 1,5 mM Mg(OAc)₂, 40 pmol 78_ppprot1_{—}087_E125′, 40 pmol 78_ppprot1_{—}087_E123′, 15 μl 3,3×rTth DNA Polymerase XLPuffer (PE Applied Biosystems), 5U rTth DNA Polymerase XL (PE Applied Biosystems).

The following conditions were used:

step 1: 5 minutes 94° C. (denaturation)

step 2: 3 seconds 94° C.(denaturation)

step 3: 2 minutes 65° C. (annealing)

step 4: 2 minutes 72° C. (elongation)

40 cycles step 2-4

step: 5: 10 minutes 72° C.

The resulting PCR fragment was cloned into the PCR cloning vector pGEM-T (Promega) as described in the instruction. The recombinant plasmid (pGEM-Teasy/087-259C-term) was sequenced to confirm the correct amplification.

### Example 29

### Demonstration of γ-Tocopherol-Methyltransferase Activity of 087-259Cterm cDNA Clone by Expression and Biochemical Analysis in E.coli

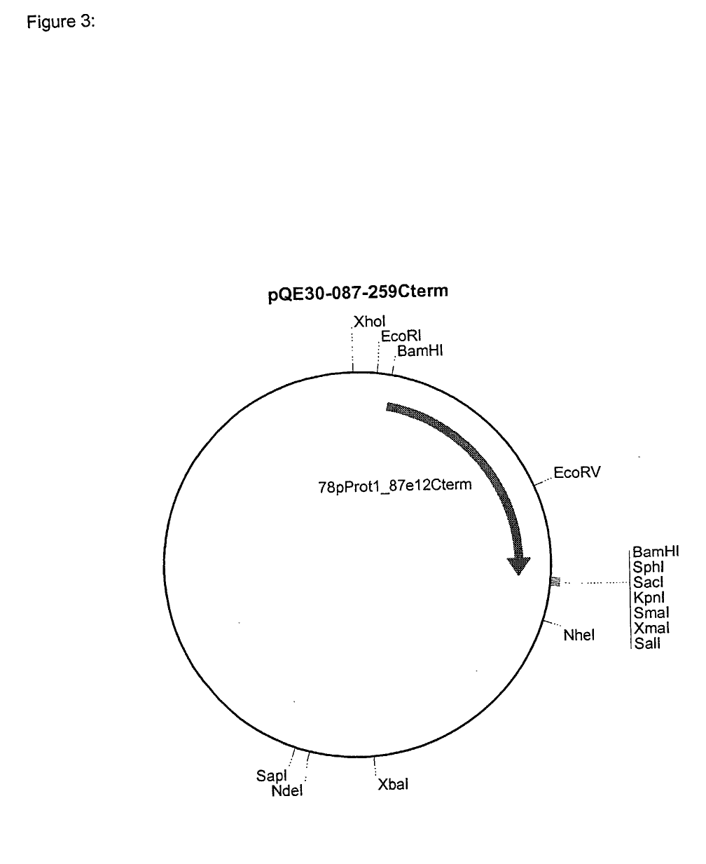

In order to demonstrate that the clone 087-259Cterm (amplified as described above) encodes a protein involved in tocopherol biosynthesis the cDNA 087-259Cterm was expressed in E.coli and tested for γ-Tocopherol methyltransferase activity. Hence, the 087-259Cterm BamHI fragment was subcloned in the correct reading frame into the BamHI site of the E. coli pQE30 expression vector (QIAexpress Kit, Qiagen). The resulting plasmid (designated pQE30-087-259Cterm, see FIG. 3) was used to transform the E.coli expression host strain M15[pREP4].

An E.coli colony transformed with the plasmid pQE30-087-259Cterm was used to inoculate an overnight culture of Luria broth containing 200 μg/ml ampicillin. In the morning an aliquot of this culture was used to inoculate a 100 ml culture of Luria broth containing 200 μg/ml ampicillin. This culture was incubated in a shaking incubator at 28° C. until the OD₆₀₀ of the culture reached 0.4, at which time isopropyl-β-D-thiogalaktopyranosid (IPTG) was added to obtain a final concentration of 0.4 mM IPTG.

The culture was incubated for additional three hours at 28° C. Afterwards the cells were harvested by centrifugation at 8000 g.

The pellet was resuspended in 600 μl lysisbuffer (approximately 1-1.5 ml/g cell pellet, 10 mM HEPES KOH pH 7.8, 5 mM Dithiothreitol (DTT), 0.24 M Sorbitol). Subsequently Phenylmethylsulfonat (PMSF) was added to a final concentration of 0.15 mM and incubated on ice for 10 minutes.

The cells were lysed by sonification with a microtip sonicator using several 10 second pulses. After adding Triton X100 (f.c. 0.1%) the homogenate was incubated for 30 minutes on ice, and subjected to centrifugation at 25000 g for 30 minutes

The supernatant of this extract was assayed for γ-tocopherol-methyltransferase activity as follows.

The γ-Tocopherol-methyltransferase assay was performed in a 500 μl volume containing 135 μl (about 300-600 μg total protein) E.coli extract expressing the 087-259 cDNA (prepared as described above), 200 μl (125 mM) Tricine-NaOH pH 7.6, 100 μl (1.25 mM) Sorbitol, 10μg (50 mM) MgCl₂ and 20 μl (250 mM) Ascorbate, 15 μl (0.46 mM ¹⁴C-methyl-S-adenosylmethionine (SAM)) as methyl group donor and 4,8 mM γ-Tocopherol as substrate. The reaction was incubated for four hours at 25° C. in the dark.

The reaction was stopped by adding 750 μl Chloroform/Methanol (1:2)+150 μl 0.9% NaCl. The tube were mixed thoroughly, the phases were separated by centrifugation and the upper part was discarded. The lower part was transferred to a new tube and vaporized under a stream of nitrogen.

The dried residue was resuspended in 20 μl ether and spotted onto a silica thin layer-chromatography (TLC) plate. The TLC plate was exposed to a phosphoimager screen.

The result shows that the in E.coli expressed 087-259Cterm protein was able to methylate γ-Tocopherol. No radioactive labelling of the substrate was observed in assays using extracts from control cells.

### Example 30

### Construction of Vectors for Expressing the Physcomitrella patens γ-Tocopherol-methyltransferase in A.thaliana and Other Plants for Altering the Content of Tocopherols

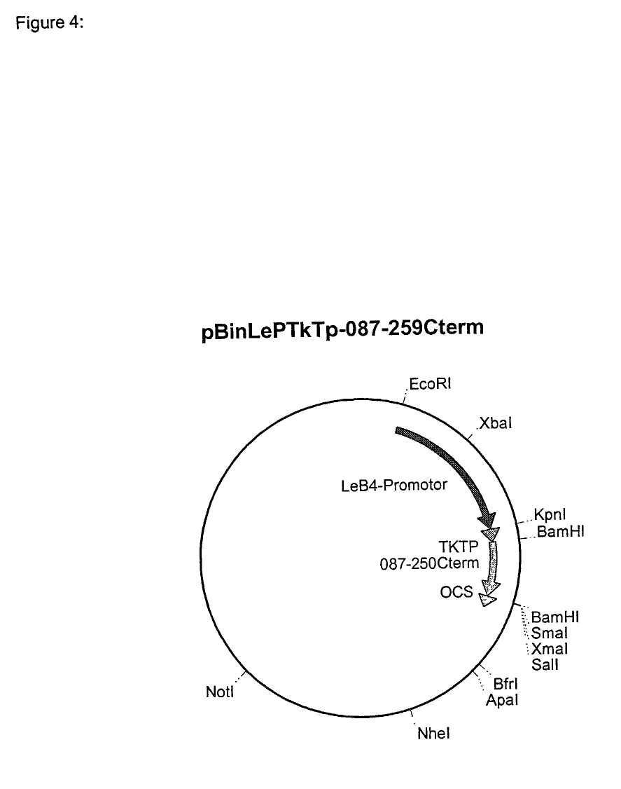

In order to manipulate the Vitamin E levels in seeds, the cDNA clone 78_ppprot1_{—}087_E12-259 encoding the Physcomitrella patens γ-tocopherol-methyltransferase was expressed under the control of a seed specific promoter in transgenic A.thaliana plants. The seed-specific plant gene expression plasmid was constructed using a pBin19 (Bevan, Nucleic Acid Research 12: 8711-8720, 1984) derivative. The plasmid contains the Vicia faba seed specific promotor from the Legumin B4 gene (Bäumlein et al., Nucleic Acids Research 14: 2707-2719, 1996), the sequence encoding the transit peptide of the N. tabacum Transketolase (TkTp) (Badur, R., Ph.D thesis, 1998, Georg August University of Göbttingen, Germany, ,“Molecular and functional analysis of isoenzymes for example of fructose-1,6-bisphosphate aldolase, phosphoglucose-isomerase and 3-deoxy-D-arabino-heptusolonate-7-phosphate synthase” [“Molekularbiologische und funktionelle Analyse von pflanzlichen Isoenzymen am Beispiel der Fructose-1,6-bisphosphat Aldolase, Phosphoglucose-Isomerase und der 3-Deoxy-D-Arabino-Heptusolonat-7-Phosphat Synthase”]) and the transcriptional termination sequence from the octopin synthase gene (Gielen et al., EMBO J. 3: 835-846, 1984). The cDNA 087-259Cterm was cloned in sense orientation as a BamHI fragment into the BamHI site of the pBin-LePTkTp9 vector. The created plasmid was designated pBinLePTkTp9-87-259Cterm. Due to the cloning in the correct reading frame the cDNA 087-259Cterm was fused to the TkTp transit peptide which governs the translocation of the 087-259Cterm protein into plastids. A recombinant plasmid designated pBin-LePTkTp9-087-259Cterm was obtained (see FIG. 4). This seed-specific 78₁₃ ppprot1_{—}087_E12-259 plant gene expression construct (pBin-LePTkTp9-087-259Cterm) was used to transform wild type A. thaliana plants.

### Equivalents

Those skilled in the art will recognize, or will be able to ascertain using no more than routine experimentation, many equivalents to the specific embodiments of the invention described herein. Such equivalents are intended to be encompassed by the following claims.

### Legends to the Figurs:

FIG. 1: Expression vector pQE30 harboring the coding sequence of full length clone 78_ppprot1_{—}092_E 12-260 resluting in vector pQE30-092-260 cds

FIG. 2: Plant transformation vector pBinLePTkTp9-092-260 cds with abbreviations as follows:

LeB4: Vicia faba legumin B4 gene promoter (2700 bp)

TKTP: Sequence encoding the N.tabacum transketolase transit peptide (245 bp)

092-260 cds: Sequence of the cDNA clone 092-260 cds (1490 bp)

OCS: Octopin synthase transcritional termination signal (219 bp)

FIG. 3: Expression vector pQE30 harboring the coding sequence of full length clone 78_ppprot1_{—087}_E12-259 resluting in vector pQE30-087-259Cterm

FIG. 4: Plant transformation vector pBinLePTkTp9-092-260 cds with abbreviations as follows:

LeB4: Vici afaba legumin B4 gene promoter (2700 bp)

TKTP: Sequence encoding the N.tabacum transketolase transit peptide (245 bp)

092-260 cds: Sequence of the cDNA clone 092-260 cds (1490 bp)

OCS: Octopin synthase transcritional termination signal (219 bp)

Table 1: Enzymes involved in production of tocopherols and/or carotenoids, the accession/entry number of the corresponding partial nucleic acid molecules, the corresponding longest clones and the position of open reading frames.

Appendix A: Nucleic acid sequences encoding for TCMRPs (Tocopherol and Caotenoid Metabolism Related protein)

Appendix B: TCMRP polypeptide sequences

1. An isolated nucleic acid molecule from a moss encoding a Tocopherol and Carotenoid Metabolism Related Protein (TCMRP), or a portion thereof.

2. An isolated nuclei acid molecule wherein the moss is selected from Physcomitrella patens or Ceratodon purpureus.

3. The isolated nucleic acid molecule of claim 1 or 2, wherein said nucleic acid molecule encodes an TCMRP capable of performing an enzymatic step involved in the production of a fine chemical.

4. The isolated nucleic acid molecule of any one of claims 1 to 3, wherein said nucleic acid molecule encodes an TCMRP capable of performing an enzymatic step involved in the metabolism of tocopherols and/or carotenoids.

5. The isolated nucleic acid molecule of any one of claims 1 to 4, wherein said nucleic acid molecule encodes an TCMRP assisting in the transmembrane transport.

6. An isolated nucleic acid molecule from mosses selected from the group consisting of those sequences set forth in Appendix A, or a portion thereof.

7. An isolated nucleic acid molecule which encodes a polypeptide sequence selected from the group consisting of those sequences set forth in Appendix B.

8. An isolated nucleic acid molecule which encodes a naturally occurring allelic variant of a polypeptide selected from the group of amino acid sequences consisting of those sequences set forth in Appendix B.

9. An isolated nucleic acid molecule comprising a nucleotide sequence which is at least 50% homologous to a nucleotide sequence selected from the group consisting of those sequences set forth in Appendix A, or a portion thereof.

10. An isolated nucleic acid molecule comprising a fragment of at least 15 nucleotides of a nucleic acid comprising a nucleotide sequence selected from the group consisting of those sequences set forth in Appendix A.

11. An isolated nucleic acid molecule which hybridizes to the nucleic acid molecule of any one of claims 1-10 under stringent conditions.

12. An isolated nucleic acid molecule comprising the nucleic acid molecule of any one of claims 1-11 or a portion thereof and a nucleotide sequence encoding a heterologous polypeptide.

13. A vector comprising one or more nucleic acid molecule(s) of any one of claims 1-12.

14. The vector of claim 13, which is an expression vector.

15. A host cell transformed with one or more expression vector(s) of claim 14.

16. The host cell of claim 15, wherein said cell is a microorganism.

17. The host cell of claim 15, wherein said cell belongs to the genus mosses or algae.

18. The host cell of claim 15, wherein said cell is a plant cell.

19. The host cell of any one of claims 15 to 18, wherein the expression of said nucleic acid molecule(s) results in the modulation of the production of a fine chemical from said cell.

20. The host cell of any one of claims 15 to 19, wherein the expression of said nucleic acid molecule(s) results in the modulation of the production of tocopherols and/or carotenoids from said cell.

21. Descendants, seeds or reproducable cell material derived from a host cell of any one of claims 15 to 20.

22. A method of producing one or more polypeptide(s) comprising culturing the host cell of any one of claims 15 to 20 in an appropriate culture medium to, thereby, produce the polypeptide.

23. An isolated TCMRP from mosses or algae or a portion thereof.

24. An isolated TCMRP from microorganisms or fungi or a portion thereof.

25. An isolated TCMRP from plants or a portion thereof.

26. The polypeptide of any one of claims 23 to 25, wherein said polypeptide is involved in the production of a fine chemical.

27. The polypeptide of any one of claims 23 to 25, wherein said polypeptide is involved in assisting in transmembrane transport.

28. An isolated polypeptide comprising an amino acid sequence selected from the group consisting of those sequences set forth in Appendix B.

29. An isolated polypeptide comprising a naturally occurring allelic variant of a polypeptide comprising an amino acid sequence selected from the group consisting of those sequences set forth in Appendix B, or a portion thereof.

30. The isolated polypeptide of any of claims 23 to 29, further comprising heterologous amino acid sequences.

31. An isolated polypeptide which is encoded by a nucleic acid molecule comprising a nucleotide sequence which is at least 50% homologous to a nucleic acid selected from the group consisting of those sequences set forth in Appendix A.

32. An isolated polypeptide comprising an amino acid sequence which is at least 50% homologous to an amino acid sequence selected from the group consisting of those sequences set forth in Appendix B.

33. An antibody specifically binding to a TCMRP of any one of claims 23 to 32 or a portion thereof.

34. Test kit comprising a nucleic acid molecule of any one of claims 1 to 12, a portion and/or a complement thereof used as probe or primer for identifying and/or cloning further nucleic acid molecules involved in the production of tocopherols and/or carotenoids or assisting in transmembrane transport in other cell types or organisms.

35. Test kit comprising an TCMRP-antibody of claim 33 for identifying and/or purifying further TCMRP molecules or fragments thereof in other cell types or organisms.

36. A method for producing a fine chemical, comprising culturing a cell containing one or more vector(s) of claim 13 or 14 such that the fine chemical is produced.

37. The method of claim 36, wherein said method further comprises the step of recovering the fine chemical from said culture.

38. The method of claim 36 or 37, wherein said method further comprises the step of transforming said cell with one or more vector(s) of claim 13 or 14 to result in a cell containing said vector(s).

39. The method of any one of claims 36 to 38, wherein said cell is a microorganism.

40. The method of any one of claims 36 to 38, wherein said cell belongs to the genus Corynebacterium or Brevibacterium.

41. The method of any one of claims 36 to 38, wherein said cell belongs to the genus mosses or algae.

42. The method of any one of claims 36 to 38, wherein said cell is a plant cell.

43. The method of any one of claims 36 to 42, wherein expression of one or more nucleic acid molecule(s) from said vector(s) results in modulation of the production of said fine chemical.

44. The method of claim 43, wherein said fine chemical is selected from the group consisting of tocopherols and carotenoids.

45. A method for producing a fine chemical, comprising culturing a cell whose genomic DNA has been altered by the inclusion of one or more nucleic acid molecule(s) of any one of claims 1-12.

46. A method of claim 45, comprising culturing a cell whose membrane has been altered by the inclusion of one or more polypeptide(s) of any one of claims 22 to 32.

47. A fine chemical produced by a method of any one of claims 36 to 46.

48. Use of a fine chemical of claim 47 or polypeptide(s) of any one of claims 22 to 32 for the production of another fine chemical.
